(19) United States
(12) Patent Application Publication
(10) Pub. No.: US 2013/0280162 A1
(43) Pub. Date: Oct. 24, 2013

(54) uPAR-ANTAGONISTS AND USES THEREOF

(71) Applicant: Nicolai Sidenius, Milano (MI) (IT)

(72) Inventor: Nicolai Sidenius, Milano (MI) (IT)

(73) Assignee: IFOM Fondazione Istituto FIRC di Oncologia Molecolare, Milano (MI) (IT)

(21) Appl. No.: 13/996,193

(22) PCT Filed: Dec. 21, 2011
(86) PCT No.: PCT/EP2011/073577

§ 371 Date: Jul. 3, 2013

Related U.S. Application Data
(60) Provisional application No. 61/426,236, filed on Dec. 22, 2010.

(51) Int. Cl. A61K 47/48; A61K 45/06

(52) U.S. Cl. CPC A61K 47/481; A61K 45/06
USPC 424/1.11; 435/188; 424/94.3

(57) ABSTRACT

The invention relates to inhibitors of the urokinase-type plasminogen activator receptor (uPAR). The generated inhibitors are bivalent uPAR-ligands containing the receptor binding domains of the extracellular protease urokinase-type plasminogen activator (uPA) and of the extracellular matrix protein vitronectin (VN), in different configurations, linked by a scaffold. The present invention also refers to the above molecules for use as a medicament, in particular for treatment of cancer, and for diagnostic purposes.

### FIELD OF THE INVENTION

The invention relates to inhibitors of the urokinase-type plasminogen activator receptor (uPAR). The generated inhibitors are bivalent uPAR-ligands containing the receptor binding domains of the extracellular protease urokinase-type plasminogen activator (uPA) and of the extracellular matrix protein vitronectin (VN), in different configurations, linked by a scaffold. The binding of such inhibitors to uPAR results in a complex where the binding sites for both uPA and VN are occupied contemporarily, thus efficiently blocking both the proteolytic and signaling activities of the receptor.

### BACKGROUND OF THE INVENTION

The urokinase plasminogen activator receptor (uPAR, also named CD87) is a membrane glycoprotein anchored to the plasma membrane by a glycosylphosphatidylinositol (GPI) anchor. Extensive in vitro, in vivo and clinical evidence suggests that uPAR plays important functions in wide range of pathological processes including tumor growth, invasion and metastasis, inflammatory diseases and viral infections. Drugs interfering effectively with uPAR-function may therefore provide novel therapeutical regimens in a variety of pathological conditions.

The molecular interaction between uPAR and its two well-established ligands, the serine protease urokinase (uPA) and the extracellular matrix (ECM) protein vitronectin (VN), is required for the activity of uPAR in the regulation extracellular proteolysis, cell adhesion, migration, invasion and proliferation (Blasi & Carmeliet, 2002; Smith & Marshall, 2010). Binding of uPA to uPAR promotes extracellular proteolysis by accelerating cell-surface plasminogen activation while the binding of VN enforces cell adhesion to the ECM enhancing migratory and proliferative signaling through integrin-dependent activation of the p130Cas and ERK1/2 signaling pathways. The importance of the interaction between uPA and uPAR in vivo has been intensively studied, and usually confirmed, using a variety of specific antagonists including antibodies, recombinant proteins, synthetic peptides as well as low molecular weight compounds. Although the importance of the interaction with VN is well documented to be crucial for the signaling activity of uPAR (Madsen et al, 2007; Smith et al, 2008), the importance of this interaction in vivo has never been addressed. In a xenograft mouse model of tumor growth using human cancer cells over-expressing different uPAR-variants the author has recently demonstrated that the interaction with VN is indeed required for the activity of uPAR in accelerating tumor growth (manuscript in preparation) supporting the assumption that this interaction is indeed a relevant anti-cancer target.

Several international applications disclose peptides ligand of urokinase receptor, such as WO01/17544. In particular, WO97/35969 discloses peptides that are capable of binding to uPAR and to inhibit the binding of an integrin and vitronectin. The document does not refer to uPA binding.

In addition, WO2008/073312 relates to urokinase-type plasminogen activator receptor epitope and monoclonal antibodies derived therefrom. The document discloses antibodies, and antigen-binding fragments thereof, specific for urokinase-type plasminogen activator receptor (uPAR) and their use for the treatment or prevention of cancer. In particular, the disclosed antibodies are specific for a particular epitope on uPAR.

WO 2005116077 identifies antibodies or other ligands specific for the binary uPA-uPAR complexes, for ternary complexes comprising uPA-uPAR and for complexes of uPAR and proteins other than uPA such as integrins. The antibodies inhibit the interaction of uPA and uPAR with additional molecules with which the complexed interact. Such antibodies or other ligands are used in diagnostic and therapeutic methods, particularly against cancer.

Tressler R J et al., (APMIS. 1999 January; 107(1):168-73) disclose urokinase receptor antagonists based on the growth factor domains of both human and murine urokinase. Such antagonists show sub-nanomolar affinities for their homologous receptors. Further modification of these molecules by preparing fusions with the constant region of human IgG has led to molecules with high affinities and long in vivo half-lives. Smaller peptide inhibitors have been obtained by a combination of bacteriophage display and peptide analogue synthesis. All of these molecules inhibit the binding of the growth factor domain of uPA to the uPA receptor and enhance binding of the uPA receptor to vitronectin.

To address the in vivo importance of the uPAR: VN-interaction in disease state, and to transfer this knowledge to the clinic, potent and specific drugs are required. The present invention describes the engineering, expression, purification and characterization of an uPAR:VN-antagonist that is about one thousand times more potent than the currently available inhibitors.

### DESCRIPTION OF THE INVENTION

The present invention concerns the conception, construction and validation of a novel type of inhibitor of the urokinase-type plasminogen activator receptor (uPAR). The generated inhibitor molecules (named uPAR-lock and uPAR-lockV2, also uPAR-lock molecules) are a bivalent uPAR-ligands containing the receptor binding domains of the extracellular protease urokinase-type plasminogen activator (uPA) and of the extracellular matrix protein vitronectin (VN) positioned in close proximity on a common scaffold. Binding of such inhibitors to uPAR results in a complex where the binding sites for both uPA and VN are occupied contemporarily and efficiently, thus blocking both the proteolytic and signaling activities of the receptor.

The inhibitor molecule of the present invention represents a potent antagonist of the physical and functional uPAR/VN-interactions and uPAR/uPA interactions.

It is very advantageous in that it targets the uPA-binding pocket in uPAR without stimulating VN-binding to receptor. Indeed, most, if not all, known drugs binding to the uPA-binding pocket of uPAR are agonists of the uPAR:VN-interaction and therefore inducing signaling. By contrast, the molecule of the instant invention uPAR-lock blocks the signaling.

In addition, there is no species-specificity barrier for the use of uPAR-lock molecules A main problem in the evaluation of drugs against the uPA:uPAR interaction is that they display profound species-specificity making it difficult to test the compound reliably in xenograft models. The specificity of uPAR-lock can easily be “set” by changing the species origin of the effector domain(s).

The present antagonist molecule displays excellent drug-properties such as long in vivo half-life due to the Fc-tag. In addition, it is constituted of human sequences, thus it is non-immunogenic. uPAR-lock molecules were designed to function as a blocking agent. Its high affinity and specificity for uPAR and the presence of the Fc-moiety make the molecule suitable to be used for uPAR-targeted diagnosis and/or therapy by for instance conjugation with appropriate effector molecules such as radionuclide, toxins etc.

Thus, the present antagonist molecule has versatile clinical applications.

It is therefore the object of the present invention a dimeric molecule comprising two polypeptides selected from the group of:
a first polypeptide comprising the growth factor-like domain of uPA (GFD domain) from aa. 11 to aa. 42 of SEQ ID No. 1, or a polypeptide encoded by the correspondent region from an uPA orthologous gene, or functional mutants or derivatives or analogues thereof; and
a second polypeptide comprising the somatomedin B domain of VN (SMB domain) from aa. 5 to aa. 39 of SEQ ID No. 2, or a polypeptide encoded by the correspondent region from a VN orthologous gene, or functional mutants or derivatives or analogues thereof;
wherein the two polypeptides are linked one to the other by a molecular scaffold.

Preferably, in the above dimeric molecule:
the first polypeptide further comprises the SMB domain from aa. 5 to aa. 39 of SEQ ID No. 2, or a polypeptide encoded by the same region from a VN orthologous gene, or functional mutants or derivatives or analogues thereof;
and
the second polypeptide further comprises the GFD domain from aa. 11 to aa. 42 of SEQ ID No. 1, or a polypeptide encoded by the same region from an uPA orthologous gene, or functional mutants or derivatives or analogues thereof.

The dimeric molecule of the invention may be obtained by any means known in the art.

A derivative may be a polypeptide with a longer or shorter sequence, i.e. modified to be resistant to enzymes, etc. . . . .

In the dimeric molecule of the invention the GFD domain preferably consists essentially of aa. 8 to aa. 48 of SEQ ID No. 1, more preferably it consists of aa. 1 to aa. 48 of SEQ ID No. 1.

In the dimeric molecule of the invention the SMB domain preferably consists of aa. 1 to 41 of SEQ ID No. 2.

In a preferred embodiment the first and the second polypeptide:
a) comprise a sequence ordered as N-terminal-SMB domain-GFD domain-C-terminal and
b) are linked to the molecular scaffold through their C-terminals.

More preferably, the SMB domain and the GFD domain are linked by a first linker peptide.

Said first linker peptide preferably consists essentially of the sequence of SEQ ID No. 3.

In the dimeric molecule of the invention each of first and second polypeptide are preferably linked to the molecular scaffold by means of a second linker peptide.

Said linker peptide preferably consists essentially of the sequence of SEQ ID No. 4.

The molecular scaffold of the dimeric molecule of the invention is preferably an immunoglobulin constant region (Fc), a leucine zipper, a chemical or a peptide linker.

In an embodiment of the invention each of heavy chain constant regions of Fc has essentially the sequence

(SEQ ID No. 5)
PKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQV
SLXaaCLVKGFYPSDIAVEWESNGQPENNYKATPPVLDSDGSFFLXaaS
KLTVDKSRWQQGNVFSCSVMHEALHNHYTQKSLSLSPGK,
wherein Xaa may be either Y or T.

In a preferred embodiment the dimeric molecule of the invention consists essentially of a first monomer of sequence of SEQ ID No. 6, and of a second monomer of sequence of SEQ ID No. 7.

In another preferred embodiment, the first and the second monomer of the dimeric molecule of the invention have the sequence of SEQ ID No. 8.

Another object of the invention is the above dimeric molecule for medical use, preferably for use as treatment of cancer.

In a preferred embodiment the dimeric molecule of the invention is conjugated to a therapeutic agent, wherein the therapeutic agent is preferably a radionuclide or a toxin.

A further object of the invention is the above dimeric molecule for use in a diagnostic method, preferably for use in the diagnosis of a uPAR-mediated pathology or tumor.

Other objects of the invention are a method of treatment of cancer comprising the administration to a subject in need thereof of a therapeutically effective amount of the dimeric molecule of the invention, a pharmaceutical composition comprising the dimeric molecule of the invention and appropriated diluents or excipients. Said pharmaceutical composition can further comprise another therapeutic agent, preferably a radionuclide or a toxin.

Another object of the invention is a kit for the diagnosis of a uPAR-mediated pathology or tumor comprising the dimeric molecule of the invention.

### DETAILED DESCRIPTION OF THE INVENTION

The invention will be now described by non limiting examples referring to the following figures:

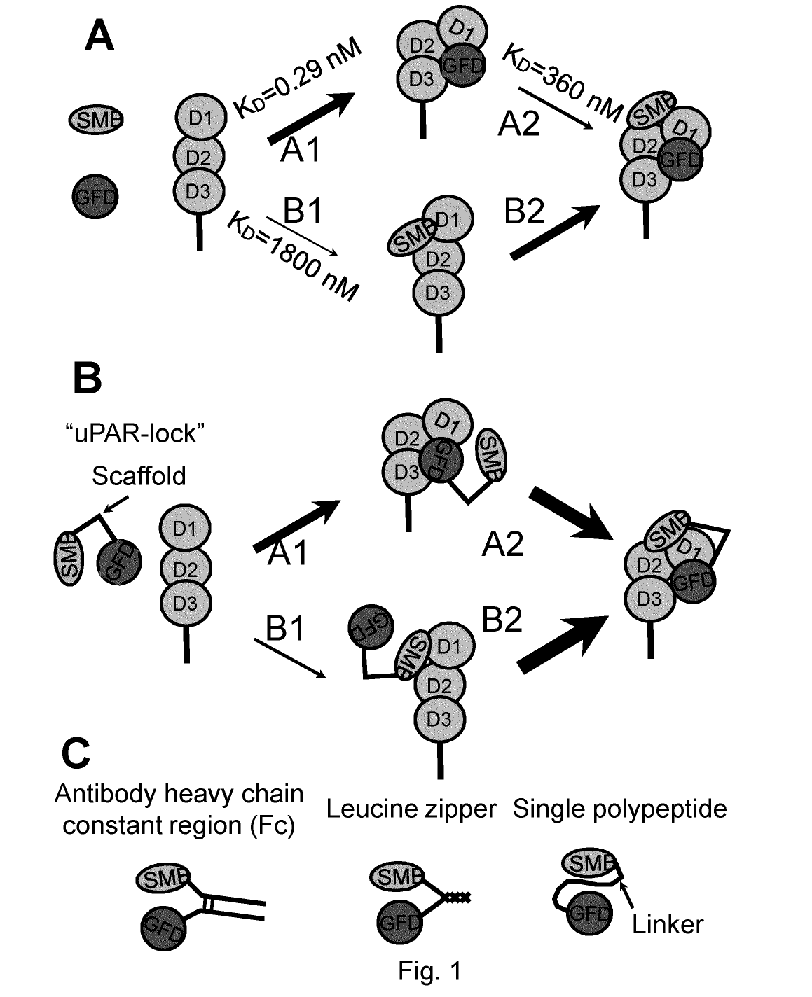

FIG. 1. Cartoon Illustrating the Forced-Proximity Concept of uPAR-Lock

The function of uPAR in extracellular proteolysis mediated by uPA-binding can be competitively inhibited by the receptor binding of the growth factor-like domain of uPA (GFD). The function of uPAR in signal transduction mediated by VN-binding can be competitively inhibited by the somatomedin B domain of VN (SMB). In the following D1, D2 and D3 are the uPAR domains.

(A) The GFD and SMB Domains as Competitive uPAR Antagonists

Blocking of uPAR-function using mixtures of isolated GFD and SMB requires two consecutive first-order inter-molecular binding reactions (1 and 2) and may follow two routes (A and B) depending on whether it is GFD or SMB that binds the receptor first. The overall stability of the ternary uPAR:GFD:SMB complex, and therefore of receptor inhibition, is limited by the weakest of the second binding reactions, which in this case is SMB-binding (KD=360 nM). Experimentally determined equilibrium constants for the discrete binding reactions, when known, are from Gårdsvoll et al. (Gardsvoll & Ploug, 2007).

(B) Forced Proximity Engineering of a Molecular Scaffold Containing Both the GFD and SMB Domains (Named “uPAR-Lock”)

When GFD and SMB are forced in close proximity by attachment to a common scaffold (uPAR-lock) receptor inhibition follows the same sequence of binding reactions. Importantly, however, the second binding reactions (A2 and B2) are now zero-order (i.e. concentration independent) intra-molecular reorganizations. In this scenario, the efficiency of receptor inhibition is thus only limited by the strongest of the initial binding reactions, which in this case is the binding of the GFD-moiety (expected KD ˜0.29 nM). Consequently, it is predicted that an uPAR-lock, made with a suitable scaffold, will be a far stronger (>1000-fold) antagonist of uPAR-function than mixtures of GFD and SMB.

(C) Scaffolds

Several different types of scaffolds can be envisaged for making an uPAR-lock. GFD and SMB molecules may be attached to the constant regions of immunoglobulin heavy chain (Fc) that form covalent dimers. The GFD and SMB domains may also be tagged with leucine zipper sequences modified to form hetero-dimers (Moll et al, 2001). Also it is possible that the GFD and SMB domains may be engineered into a single polypeptide using appropriate linker regions.

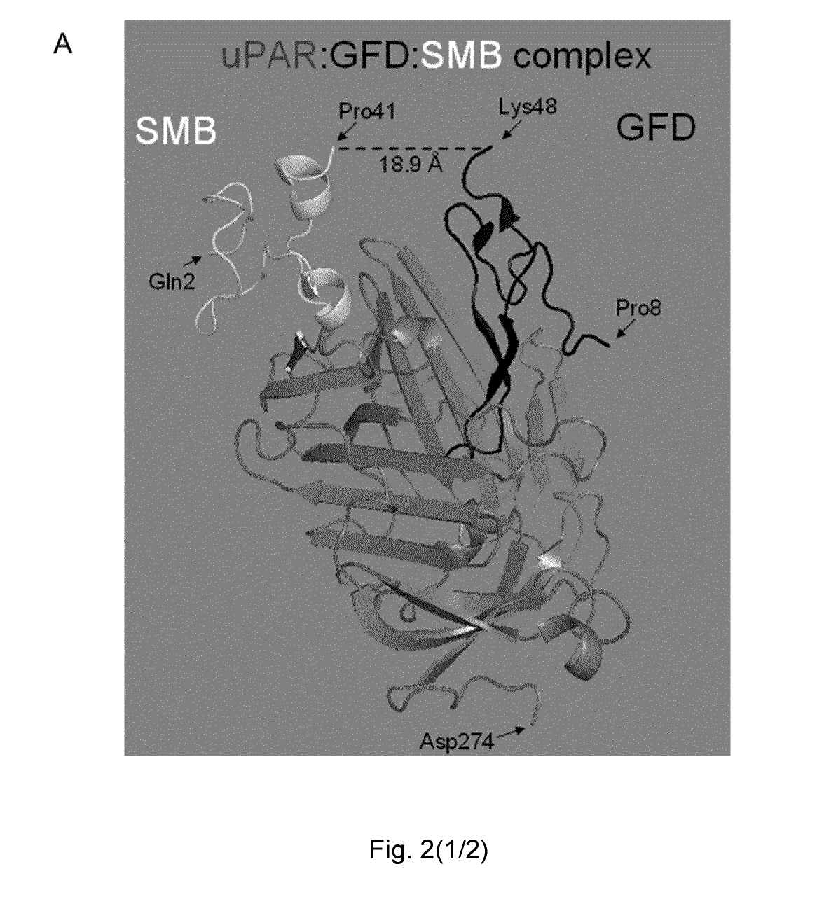

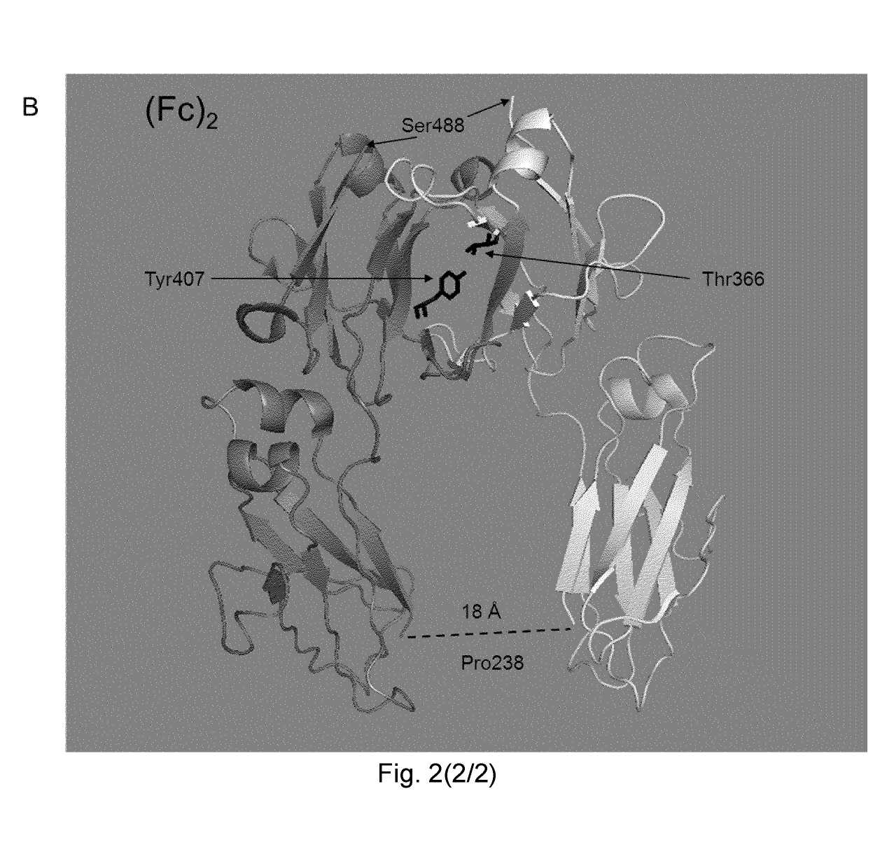

FIG. 2. Crystal Structure of the Ternary uPAR:GFD:SMB-Complex and a Human Immunoglobulin Constant Region.

(A) Crystal structure of the ternary complex between uPAR (in grey), the receptor binding domain of uPA (GFD, in black) and the receptor binding domain of VN (SMB, in white). N-terminal residues of GFD and SMB (Gln2 and Pro8) and C-terminal residues of GFD, SMB and uPAR (Lys48, Pro41, Asp274) are indicated. Note that the C-terminal residues of GFD (Lys48) and of SMB (Pro41) are distant only 18.9 Å and have the same polarity pointing away from the receptor and away from the presumed membrane anchorage location highlighted by the C-terminal residue of uPAR (Asp274) in the structure.

(B) Crystal structure of a dimeric human IgG heavy-chain constant region (Fc)₂. N-terminal residue (bottom figure, Pro238) and C-terminal residue (top figure Ser444). Note that the N-terminal residues of the two polypeptides are spaced similarly to the C-terminal residues of GFD and SMB bound to uPAR. Two residues (Tyr407 and Thr366, in black) that may be manipulated to favor hetero-dimerization (Ridgway et al, 1996) are shown in the right and left side of the figure. The structures are from protein database (PDB) entries 3BT2 and 1H3X and were elaborated with the MacPyMOL software.

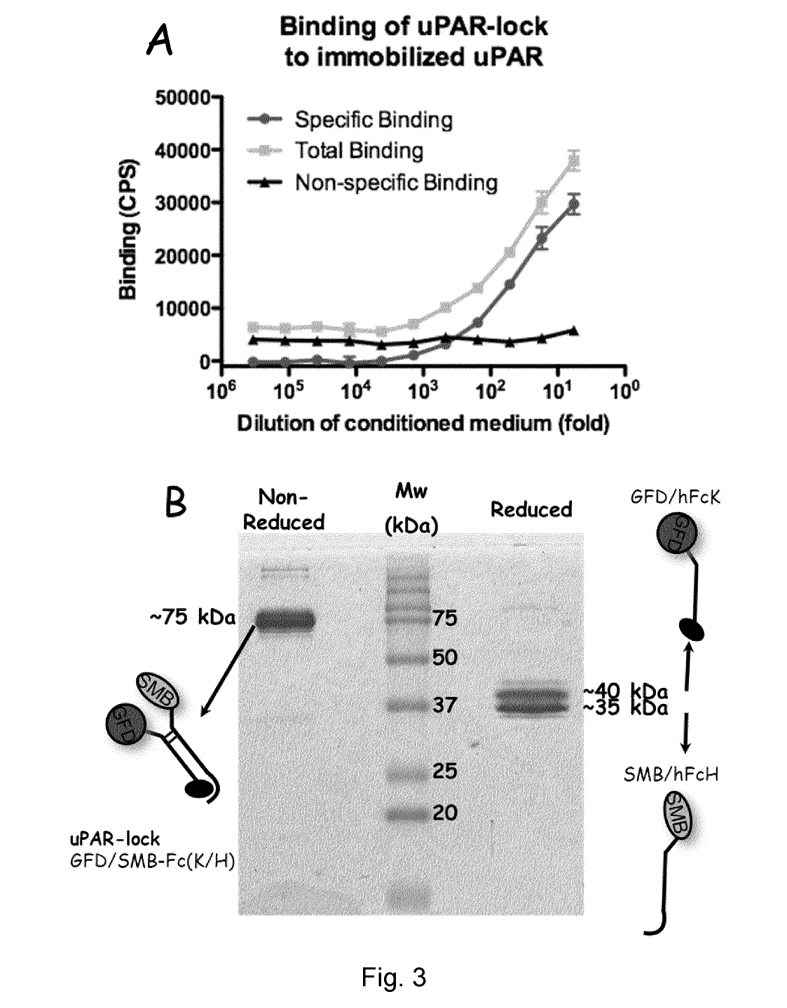

FIG. 3. (A) the Conditioned Medium of uPAR-Lock Transfected Cells Display uPAR Binding Activity.

96-well plates coated with soluble uPAR (5 nM) were incubated with serial dilutions of the conditioned medium from Phoenix cells co-transfected with GFD/FcK and SMB/FcH (i.e. uPAR-lock). Bound uPAR was detected using secondary reagents detecting human Fc. Total binding to uPAR coated wells (squares), non-specific binding to uncoated wells (triangles) and specific binding (circles) are shown.

(B) Cartoon of the Predicted uPAR-Lock Structure and the Appearance of the Purified Protein by SDS-PAGE.

uPAR-lock is a covalent (disulfide bonded) hetero-dimer between GFD/FcK (FcK=Fc “Knob”=Fe carrying a Thr366->Tyr substitution) and SMB/FcH (FcH=Fc “Hole”=Fc carrying a Tyr407->Thr substitution). 3 μg of purified protein was separated by 12% SDS-PAGE under non-reducing (left) and reducing (right) conditions and the gel stained with colloidal Comassie stain.

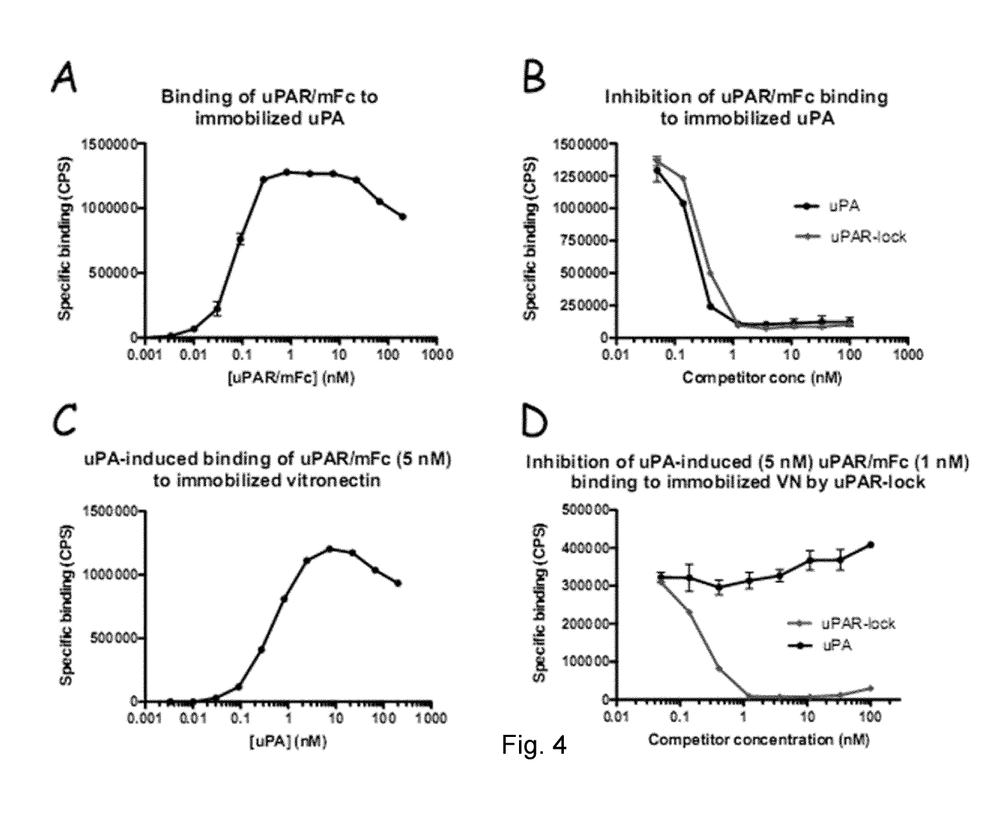

FIG. 4. Antagonistic Properties of uPAR-Lock.

(A) Fc-Tagged uPAR Bind with High Affinity to Immobilized uPA.

Immobilized uPA was incubated with increasing concentrations of uPAR tagged with a mouse Fc (uPAR/mFc). After washing bound uPAR/mFc was quantified using a biotinylated anti-mouse Fc antibody and Europium labeled streptavidin. Note that uPAR/mFc display specific high-affinity binding to immobilized uPA.

(B) uPAR-Lock Inhibit uPAR Binding to Immobilized uPA.

Immobilized uPA was incubated with uPAR/mFc (1 nM) in the presence of increasing concentrations of uPAR-lock (diamonds) or uPA (circles). The amount of uPAR/mFc bound to the immobilized uPA was quantified as above. Note that both uPAR-lock and uPA are competitive antagonists of uPAR/mFc binding to immobilized uPA.

(C) High-Affinity Binding of uPAR to Immobilized VN Requires uPA.

Immobilized VN was incubated with uPAR/mFc (5 nM) mixed with increasing concentrations of uPA. After washing, bound uPAR/mFc was quantified as above. Note that uPA is required for uPAR/Fc binding to immobilized VN.

(D) uPAR-Lock Inhibit uPA-Induced uPAR Binding to VN.

Immobilized VN was co-incubated with uPAR/mFc (1 nM), uPA (5 nM) and increasing concentrations of uPAR-lock (diamonds) or uPA (circles). After washing bound uPAR/mFc was quantified as above. Note that uPAR-lock, in contrast to uPA, is an antagonist of uPAR binding to immobilized VN.

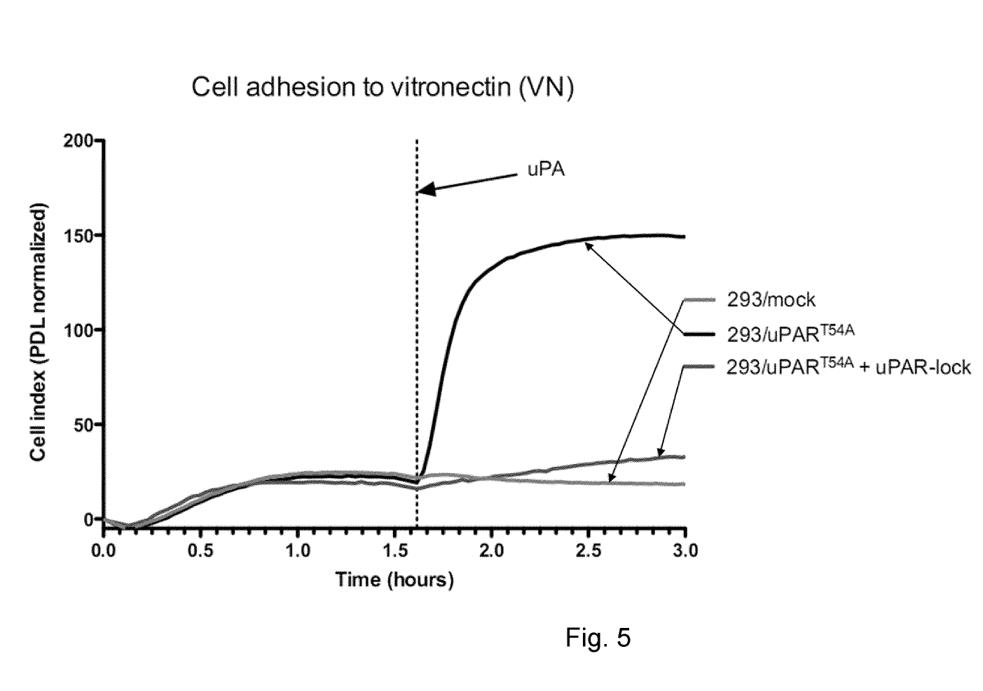

FIG. 5. uPAR-Lock Specifically Inhibits uPAR-Mediated Cell Adhesion to VN.

Mock-transfected and 293/uPAR^{T54A} cells in the presence or absence of 50 nM uPAR lock were seeded (20.000/well) in 96-well E-plates coated with VN. Cell adhesion was quantified by measuring the impedance (cell index) every two minutes for 3 hours in a real time cell analyzer (RTCA) instrument. When basal cell adhesion (mediated by the αᵥβ₅ integrin, (Madsen et al, 2007)) reached plateau the wells were added uPA to a final concentration of 10 nM to induce uPAR^{T54A} binding to VN and the recordings continued. Note that uPAR-lock completely inhibits the uPA-induced increase of cell adhesion of 293/uPAR^{T54A} cells (compare the curves after uPA-addition). uPAR-lock does not affect integrin mediated adhesion (compare curves before uPA-addition) and uPA does not modulate the adhesion of cells transfected with empty pcDNAS/FRT-TO vector (293/mock).

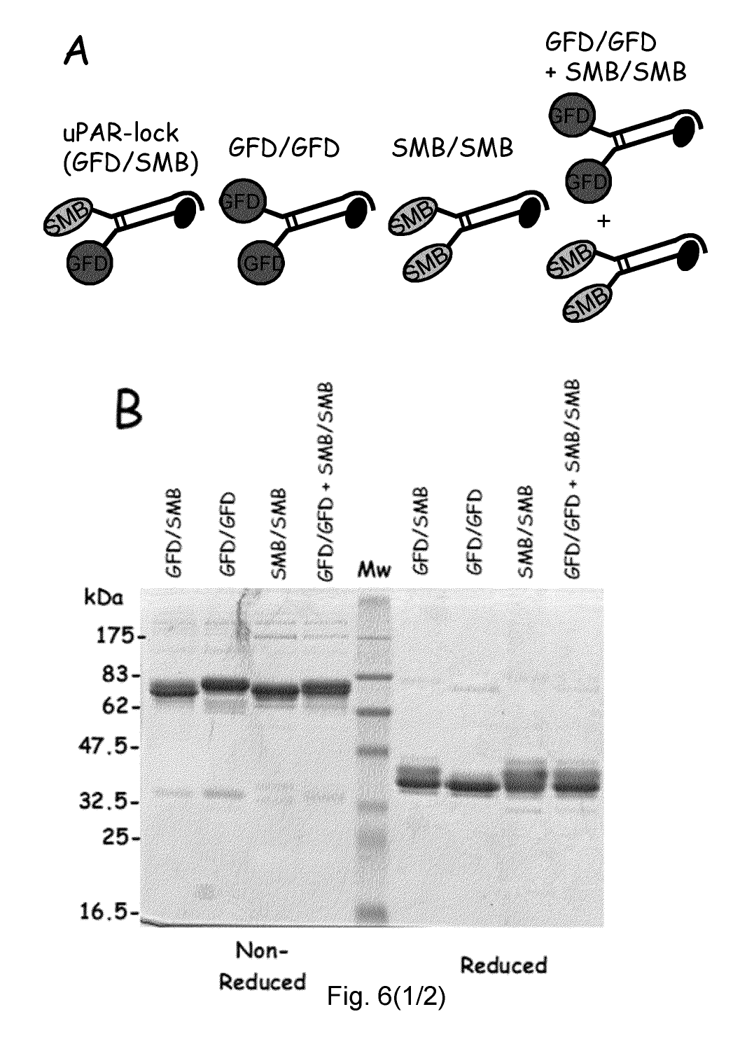

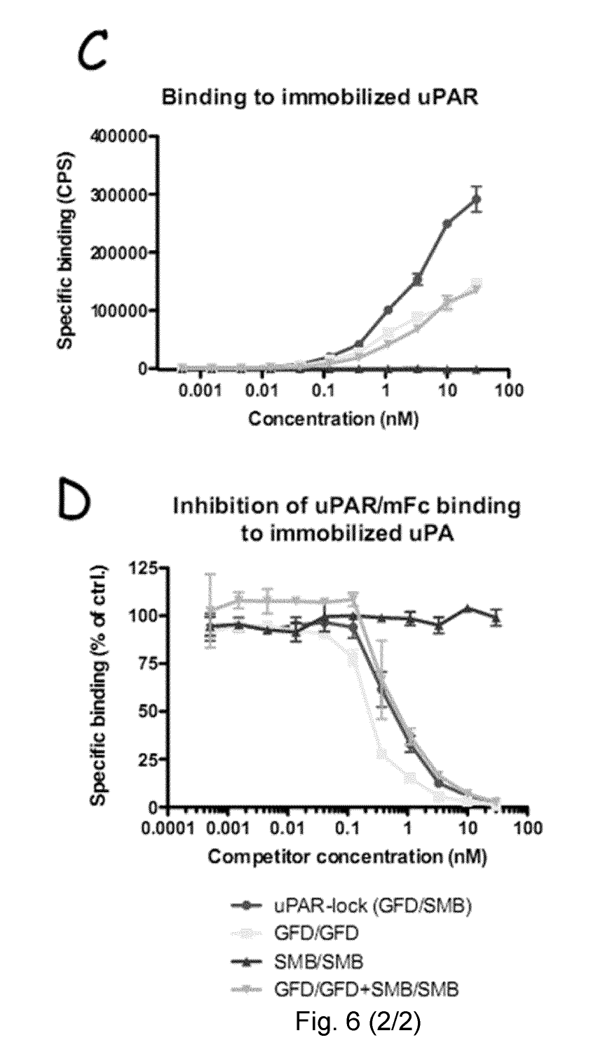

FIG. 6. (A) Cartoon of the uPAR-Lock and Control Variants.

As described above uPAR-lock is hetero-dimer between Fc-tagged GFD and SMB domains. The GFD/GFD and SMB/SMB hybrids are constructed in the same way as uPAR-lock but has either GFD or SMB domains on both polypeptides. For some experiments a mixture of GFD/GFD and SMB/SMB were used as this has the same domain composition as uPAR-lock but with the GFD and SMB domains located on separate scaffolds.

(B) Physical appearance of uPAR-lock and control proteins. Three micrograms of the purified proteins was separated by 10% SDS-PAGE under non-reducing (left) and reducing (right) conditions and the gel stained with colloidal Comassie stain.

(C) Increasing concentrations of uPAR-lock (circles), GFD/GFD (squares), SMB/SMB (triangles tip up) and GFD/GFD+SMB/SMB (triangles tip down) were allowed to bind to immobilized uPAR. Bound protein was detected using secondary reagents detecting human Fc. Note that the strongest binding is seen with uPAR-lock.

(D) Inhibition of uPAR/mFc binding to immobilized uPA by uPAR-lock and control proteins. Immobilized uPA was incubated with mixtures of uPAR/mFc (1 nM) and increasing concentrations of uPAR-lock and bound uPAR/mFc quantified using the assay described in FIG. 4B. Note that all preparations containing the GFD domain act as competitive uPAR/uPA-interaction antagonists.

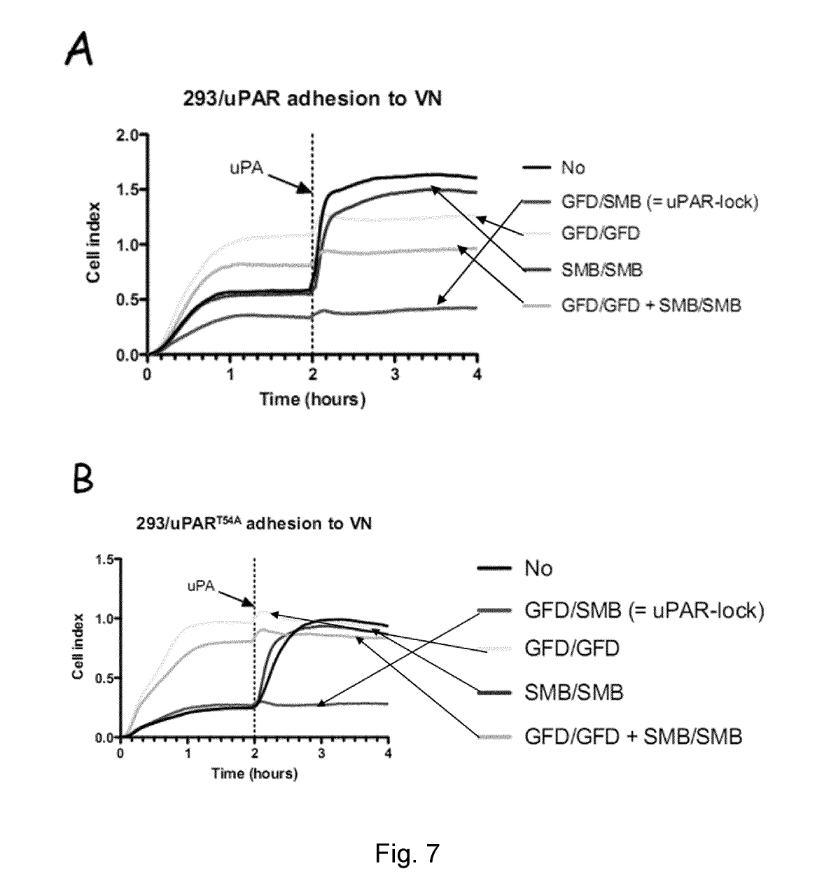

FIG. 7. Forced Proximity Between GFD and SMB is Required for the Inhibitory Activity of uPAR-Lock on uPAR-Mediated Cell Adhesion to VN.

293/uPAR (A) and 293/uPART54A (B) cells (20.000/well) were seeded in a VN-coated 96-well E-plate in the absence (black) or presence of uPAR-lock (red), GFD/GFD (yellow), SMB/SMB or GFD/GFD+SMB/SMB (blue) and allowed to adhere. Two hours after seeding wells were added uPA to 10 nM and cell adhesion measurements continued for another two hours. Note that uPAR-lock inhibit uPA-independent uPAR-mediated cell adhesion to VN (compare red and black curves before uPA-addition in panel 7A) as well as uPA-induced adhesion (compare red and black curves after uPA-addition in panels 7A and 7B). In contrast both GFD/GFD and GFD/GFD+SMB/SMB are strong agonists of uPAR-mediated VN adhesion (compare yellow and green with black curves before uPA-addition in panel 7A and 7B). SMB/SMB is largely inactive.

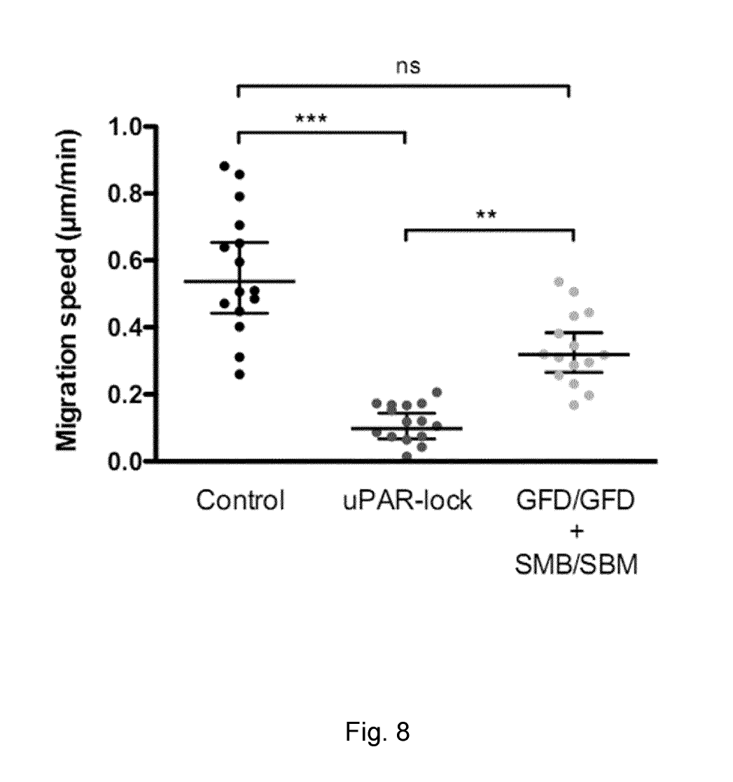

FIG. 8. uPAR-Lock Inhibits uPAR-Mediated Cell Migration and Forced Proximity Between GFD and SMB is Required.

293/uPAR cells were seeded in 12-well plates in complete serum containing medium and allowed to adhere over night. The next day the medium was replaced with complete medium without (left) or containing uPAR-lock (center) or GFD/GFD+SMB/SMB (both at 20 nM, right) and transferred to a time-lapse microscope. Random cell migration was recorded as previously described (Madsen et al, 2007) and quantified by manual cell tracking using the software ImageJ. Each dot represents a single cell. Mean migration speeds (+/−95% confidence intervals) are shown. The data were analyzed by non-parametrical analysis and corrected for multiple comparisons (*** P<0.001 **P<0.01 and ns P>0.05). Note that uPAR-lock dramatically inhibits the migration of 293/uPAR cells.

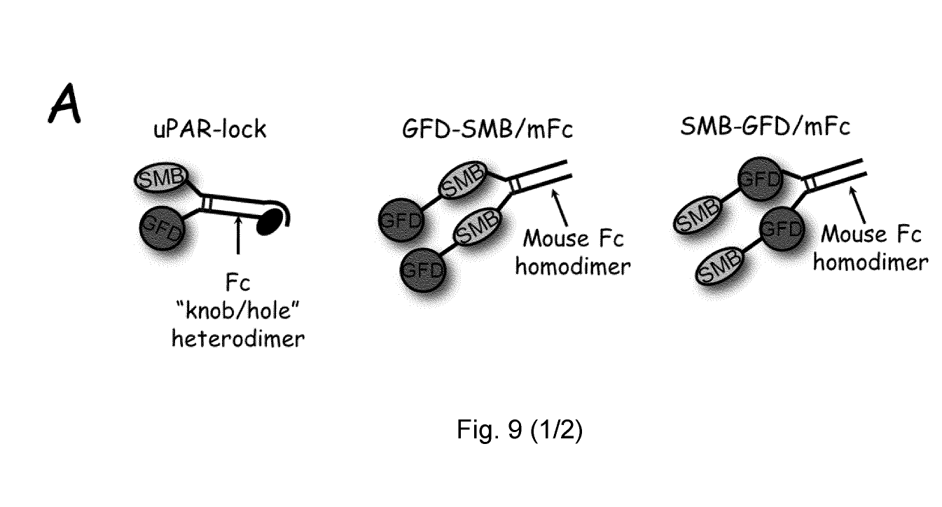

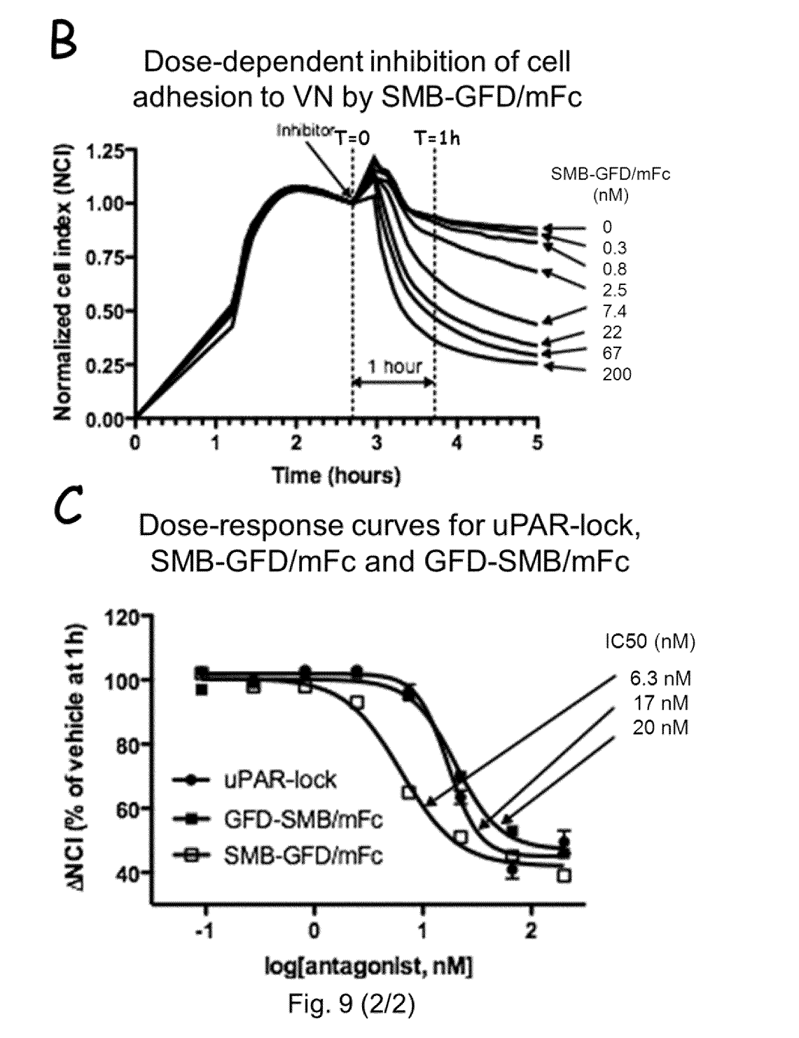

FIG. 9. Inhibitory Activities of Homodimeric uPAR-Lock Variants.

(A) Cartoon illustrating the structure of the homodimeric uPAR-lock variants containing GFD and SMB domains within a single polypeptide chain. As described above, uPAR-lock is a disulfide-linked heterodimer with the GFD and SMB located on two different polypeptides and tagged with human Fc constant regions containing Knob and hole mutations to favor heterodimerization. In contrast, GFD-SMB/mFc and SMB-GFD/mFc are homodimers containing both the SMB and GFD domains on the same polypeptide chain. (B) 293 cells expressing human uPAR were seeded in an 96-well E-plates coated with VN and transferred to a real time cell analyzer instrument (RTCA, xCELLigence, SP Roche Corp.). The electric impedance (termed cell index, CI), a measure of cell adhesion, was recorded at regular intervals. When the CI reached a plateau, indicative of complete cell adhesion, the wells were added a 3-fold dilution curve of SMB-GFD/mFc to the final concentrations indicated in the graph. The E-plate was returned to the instruments and the impedance measurements continued at regular intervals. The time point at which the inhibitor were added (T=0) and the time point (T=1 h) used for calculation of dose response curves shown in panel C are indicated by stippled vertical lines. The curves show the normalized cell index (NCI, Y-axis) as a function of time (X-axis). All cell indexes were normalized to the cell index measured immediately prior to inhibitor addition. (C) To determine IC50 values, the NCI measured one hour after reagent addition were calculated in % of the NCI for vehicle treated cells at the same time point (ΔNCI, Y-axis) and graphed in function of inhibitor concentration (X-axis). Sigmoidal dose response curves (variable slope) were fitted using the Prism 5 software suite and their derived IC50 values are indicated.

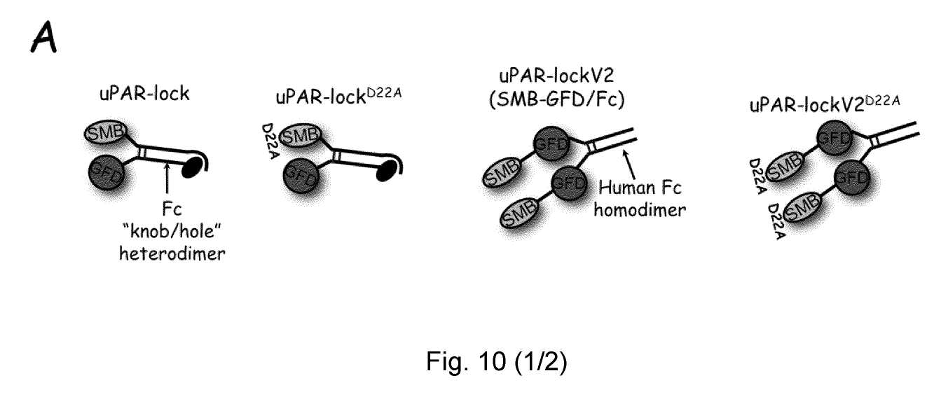

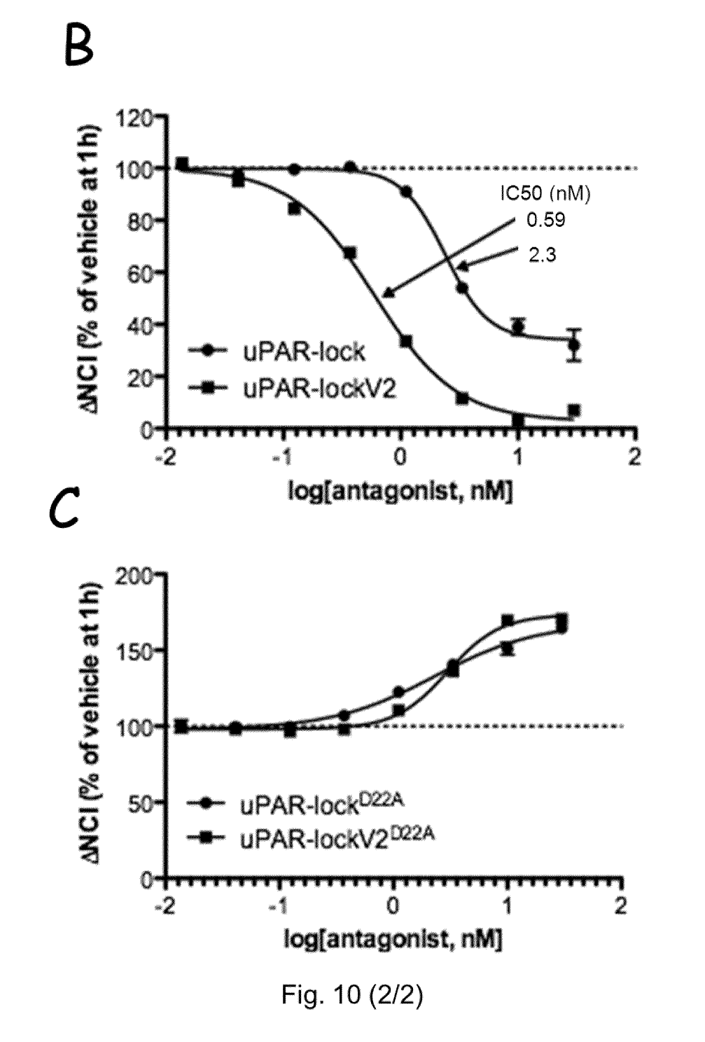

FIG. 10. uPAR-LockV2 Activity and Proof of Principle

(A) Cartoon illustrating the structure of the heterodimeric uPAR-lock and homodimeric uPAR-lockV2, as well as variant of these carrying a single amino acid substitution (D22A) in the SMB domain that impairs the interaction of this domain with uPAR (Okumura Y, et al. J Biol Chem 2002). uPAR-lockV2 is identical to the SMB-GFD/mFc shown in FIG. 9A with the only exception that the constant region (Fc) in uPAR-lockV2 is derived from a human IgG. (B) 293 cells expressing human uPAR were seeded in an 96-well E-plates coated with VN(1-66)/Fc^{RAD} and transferred to a real time cell analyzer instrument (RTCA, xCELLigence, SP Roche Corp.). Cells were treated with a dilution curve of uPAR-lock and uPAR-lockV2 and dose response curves calculated as shown in FIG. 9. (C) Same experiment as in panel B using the D22A substituted uPAR-lock variants. Note that these variants stimulate adhesion.

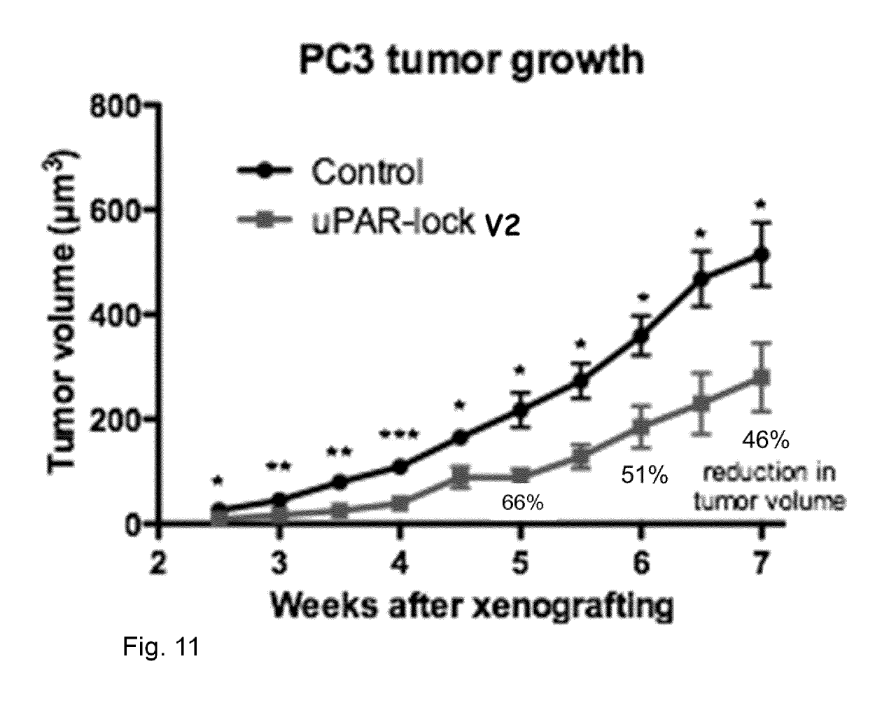

FIG. 11. Inhibition of Tumor Growth In Vivo by uPAR-LockV2.

Effect of uPAR-lockV2 on prostate cancer growth in vivo. Male Balb C nu/nu mice were inoculated with (1×10⁶) PC-3 cells through the s.c. route. Animals were treated with vehicle (PBS), 10.0 mg/kg of control mouse immunoglobulin or uPAR-lockV2 through the i.p. route. Tumors were measured twice weekly, and tumor volume was determined as described in Materials and Methods. Significant differences from control are represented by asterisks (*P<0.05, **P<0.01 and ***P<0.001).

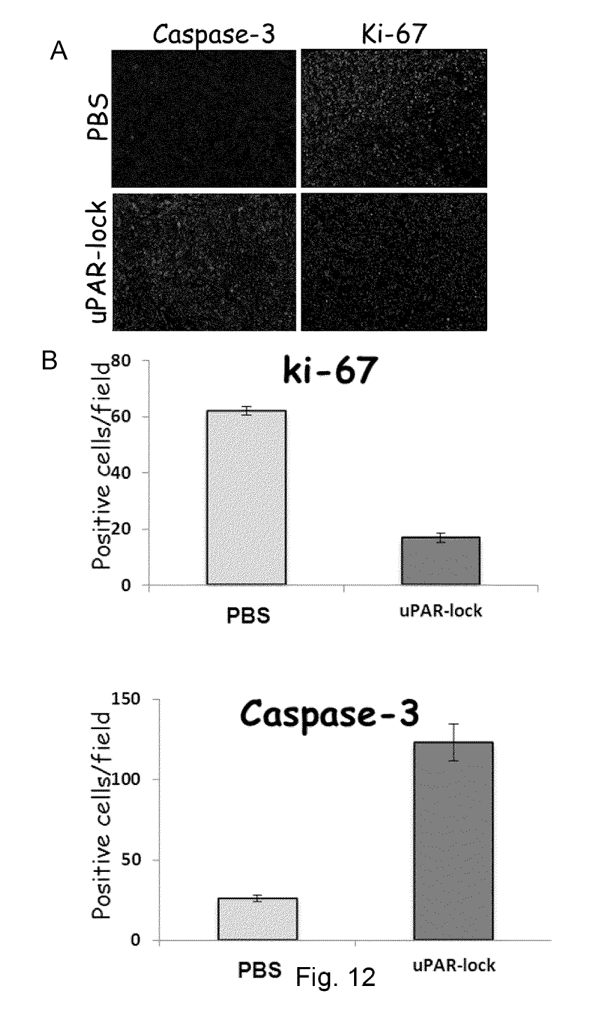

FIG. 12. uPAR-Lock Reduces PC-3 Tumor Cell Proliferation and Promotes Apoptosis In Vivo

Male athymic nu/nu mice were inoculated subcutaneously with PC-3 cells and treated by bi-weekly injections with PBS or 10.0 mg/kg of uPAR-lockV2 via intraperitoneal route. Eight weeks after xenografting, the tumors were harvested and subjected to immunohistochemical analysis (Panel A) as described in the materials and methods section. Ki-67 and Caspase-3 stainings are shown and nuclei are counterstained with Dapi. Quantification of the data is shown in Panel B.

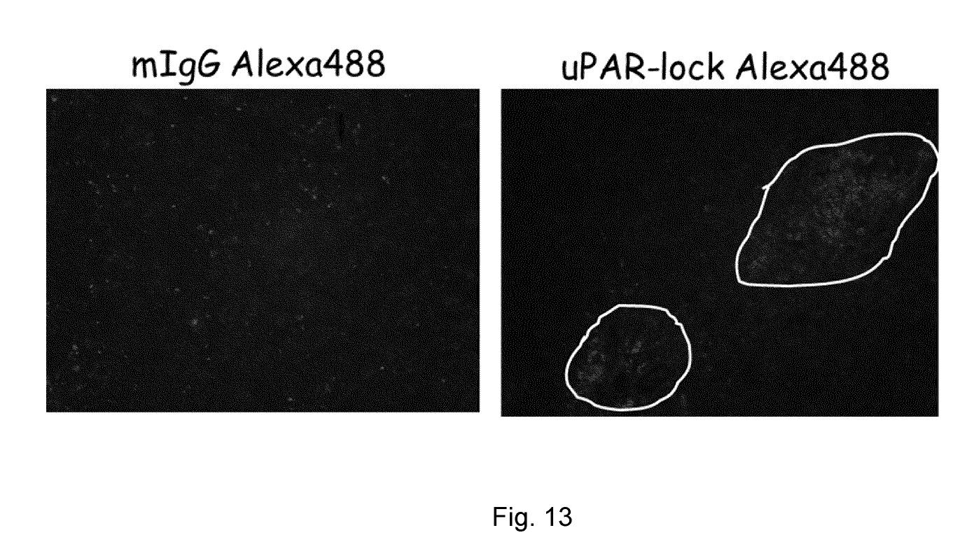

FIG. 13. Tumor Targeting Using uPAR-Lock

Mice carrying PC-3 tumors were injected with Alexa488 labeled uPAR-lockV2 or Alexa488 labeled mouse IgG and 24 hours later tumors were excised and analyzed by fluorescent microscopy. Evident areas of fluorescence can be observed in tumors from animals injected with labeled uPAR-lockV2 (outlined areas in right panel) while similar areas are not observed in tumors from mice treated with labeled mouse IgG. Representative micrographs are shown.

### EXPERIMENTAL PROCEDURES

### Amino Acid Sequences of uPA, VN and uPAR

Amino acid sequence of human uPA with the signal peptide (Met⁻²⁰-Gly⁻¹) in cursive, the mature protein (Ser¹-Leu⁴¹¹) in bold and the uPAR-binding growth factor-like domain (GFD, Ser1-Lys48) in bold/underlined:

(SEQ ID No. 9)
MRALLARLLLCVLVVSDSKGSNELHQVPSNCDCLNGGTCVSNKYFSNIHWCNCPKKFGGQHC
EIDKSKTCYEGNGHFYRGKASTDTMGRPCLPWNSATVLQQTYHAHRSDALQLGLGKHNY
CRNPDNRRRPWCYVQVGLKPLVQECMVHDCADGKKPSSPPEELKFQCGQKTLRPRFKIIG
GEFTTIENQPWFAAIYRRHRGGSVTYVCGGSLISPCWVISATHCFIDYPKKEDYIVYLGRSR
LNSNTQGEMKFEVENLILHKDYSADTLAHHNDIALLKIRSKEGRCAQPSRTIQTICLPSMYN
DPQFGTSCEITGFGKENSTDYLYPEQLKMTVVKLISHRECQQPHYYGSEVTTKMLCAADPQ
WKTDSCQGDSGGPLVCSLQGRMTLTGIVSWGRGCALKDKPGVYTRVSHFLPWIRSHTKE
ENGLAL

Amino acid sequence human VN with the signal peptide (Met⁻¹⁹-Ala⁻¹) in cursive, the mature protein (Asp¹-Leu⁴⁵⁹) in bold and the uPAR-binding somatomedin-B domain (SMB, Asp¹-Pro⁴¹) bold/underlined:

(SEQ ID No. 10)
MAPLRPLLILALLAWVALADQESCKGRCTEGFNVDKKCQCDELCSYYQSCCTDYTAECKPQV
TRGDVFTMPEDEYTVYDDGEEKNNATVHEQVGGPSLTSDLQAQSKGNPEQTPVLKPEEE
APAPEVGASKPEGIDSRPETLHPGRPQPPAEEELCSGKPFDAFTDLKNGSLFAFRGQYCYEL
DEKAVRPGYPKLIRDVWGIEGPIDAAFTRINCQGKTYLFKGSQYWRFEDGVLDPDYPRNIS
DGFDGIPDNVDAALALPAHSYSGRERVYFFKGKQYWEYQFQHQPSQEECEGSSLSAVFEHF
AMMQRDSWEDIFELLFWGRTSAGTRQPQFISRDWHGVPGQVDAAMAGRIYISGMAPRPS
LAKKQRFRHRNRKGYRSQRGHSRGRNQNSRRPSRATWLSLFSSEESNLGANNYDDYRMD
WLVPATCEPIQSVFFFSGDKYYRVNLRTRRVDTVDPPYPRSIAQYWLGCPAPGHL

Amino acid sequence of human uPAR with the signal peptide (Met⁻²²-Gly⁻¹) in cursive, a C-terminal peptide (Ala²⁸⁴-Thr³¹³) removed during synthesis upon addition of the glycolipid membrane anchor attached to Gly²⁸³ in cursive and underlined, and the mature protein (Leu¹-Gly²⁸³) in bold:

(SEQ ID No. 11)
MGHPPLLPLLLLLHTCVPASWGLRCMQCKTNGDCRVEECALGQDLCRTTIVRLWEEGEELEL
VEKSCTHSEKTNRTLSYRTGLKITSLTEVVCGLDLCNQGNSGRAVTYSRSRYLECISCGSSD
MSCERGRHQSLQCRSPEEQCLDVVTHWIQEGEEGRPKDDRHLRGCGYLPGCPGSNGFHNN
DTFHFLKCCNTTKCNEGPILELENLPQNGRQCYSCKGNSTHGCSSEETFLIDCRGPMNQCLV
ATGTHEPKNQSYMVRGCATASMCQHAHLGDAFSMNHIDVSCCTKSGCNHPDLDVQYRSG
AAPQPGPAHLSLTITLLMTARLWGGTLLWT

### Expression Vector Construction.

The expression vectors for Fc-tagged SMB and GFD are based on the pFRT/TO-Fc plasmid (Madsen et al, 2007) however a number of modifications were introduced to facilitate the shuffling of different coding regions as well as to improve protein yield and allow for the removal of the Fc-tag from the recombinant proteins by specific protease cleavage. Firstly, an XhoI restriction site located in the vector sequence downstream of the Fc coding region was destroyed by site-directed mutagenesis using oligos dXu/dXd. Secondly, a linker encoding a cleavage sequence for the PreScission protease made by annealing oligos PreF/PreR was inserted in the XhoI site located at the signal peptide/Fc junction. To remove the introns present in the Fc region of the construct, which was found to increase the yield of recombinant protein, the vector was transfected into CHO cells, RNA extracted, reverse transcribed, and the cDNA amplified with oligos hVNukpn/FcNr. The PCR product was digested Kpn1/NotI and used to replace the corresponding fragment of the parental vector generating pFRT/TO-Fc. For transient protein expression the Fc cassette was transferred KpnI/NotI to the pEGFP-N1 vector (Clontech Inc.) generating pN1-Fc. Knob and hole mutations (T366Y and Y407T, (Ridgway et al, 1996)) were introduced in the Fc regions by site-directed mutagenesis using oligo pairs FcKnobF/FcKnobR and FcHoleF/FcHoleR yielding vectors pN1-FcK and pN1-FcH, respectively. Sequences encoding the signal peptide (the negative amino acids refers to signal peptide sequence) and SMB domain of VN (Met⁻¹⁹ to Pro⁴¹, MAPLRPLLILALLAWVALADQESCKGRCTEGFNVDKKCQCDELCSYYQSCCTDYTAEC KP (aa 1-60 of SEQ ID No. 10), signal peptide in cursive were generated by amplifying a VN cDNA with oligos hVNukpn/SMBRV2 and cloned KpnI/XhoI in pN1-FcK and pN1-FcH. A sequence encoding the signal peptide and GFD domain of uPA (amino acids Met⁻²⁰ to Lys⁴⁸, MRALLARLLLCVLVVSDSKGSNELHQVPSNCDCLNGGTCVSNKYF SNIHWCNCPKKFG GQHCEIDKSK (aa 1-68 of SEQ ID No. 9), signal peptide in cursive was generated by amplification of a human uPA cDNA using oligos ATFkpnF/GFDRV and cloned as described for the SMB. This cloning strategy generates mature fusion proteins composed of GFD (residues 1-48, (SEQ ID No. 1)) or SMB (residues 1-41, (SEQ ID No. 2)) connected to hinge and Fc regions of a human IgG1 by a GSGLELEVLFQGPIE (SEQ ID No. 4) linker. The expression vector for Fc-tagged uPAR was generated by amplification of a human uPAR cDNA with oligos URfSK/uPARXd and cloning the product KpnI/XhoI in pFRT/TO-Fc. The expression vector for uPAR tagged with a murine immunoglobulin heavy chain constant region (mFc) was generated by assembling (uPAR cDNA, amplified URfSK/UpreR2D, digested KpnI/XhoI) and an IgG1 cDNA (clone IRAVp968B035D, obtained from imaGenes GmbH, amplified mFcU/mFcD, digested XhoI/NotI) in pEGFP-N1 (digested KpnI/XhoI). The resulting mature chimeric protein (uPAR/mFc) is composed of human uPAR residues 1-277, a LEVLFQGPLEAGAG (SEQ ID No. 36) linker and amino acids 216-441 of the mouse immunoglobulin heavy chain (numbered according to (Adetugbo, 1978)). The expected sequence of all coding regions was confirmed by sequencing.

Oligonucleotide sequences:
dXu
(SEQ ID No. 12)
5′-gtaaatgagcggccgcgtcgagtctagaggg-3′
dXd
(SEQ ID No. 13)
5′-ccctctagactcgacgcggccgctcattta-3′
PreF
(SEQ ID No. 14)
5′-tcgagctggaagttctgttccaggggccca-3′
PreR
(SEQ ID No. 15)
5′-agctacccggggaccttgtcttgaaggtcg-3′
hVNukpn
(SEQ ID No. 16)
5′-cggggtaccatggcacccctgaga-3′
FcNr
(SEQ ID No. 17)
5′-ttgcggccgctcatttacccggagacag-3′
FcKnobF
(SEQ ID No. 18)
5′-aaccaggtcagcctgtactgcctggtcaaaggc-3′
FcKnobR
(SEQ ID No. 19)
5′-gcctttgaccaggcagtacaggctgacctggtt-3′
FcHoleF
(SEQ ID No. 20)
5′-ggctccttcttcctcaccagcaagctcaccgtg-3′
FcHoleR
(SEQ ID No. 21)
5′-cacggtgagcttgctggtgaggaagaaggagcc-3′
ATFkpnF
(SEQ ID No. 22)
5′-gcggtacccgccaccatgagagccctgctggcgcgc-3′
GFDRV
(SEQ ID No. 23)
5′-gcctcgagtcctgatccttttgacttatctatttcaca-3′
SMBRV2
(SEQ ID No. 24)
5′-gcctcgagtcctgatccgggcttgcactcagccgt-3′
URfSK
(SEQ ID No. 25)
5′-gcgtcgacggtacccgccaccatgggtcacccgccgctgctg-3′
uPARXd
(SEQ ID No. 26)
5′-agcctcgagcccactgcggtactggacatc-3′
UpreR2D
(SEQ ID No. 27)
5′-gcctcgaggggcccctggaacagaacttccagatccaggtctgggt
ggttacagccac-3′
mFcU
(SEQ ID No. 28)
5′-gcctcgaggcaggagcaggacccagggattgtggttgtaa-3′
mFcD
(SEQ ID No. 29)
5′-gcgcggccgctcatttaccaggagagtg-3′

Composition of uPAR-Lock

The composition of uPAR-lock constructed as described above is a molecule, in particular a disulphide-linked heterodimer composed of the two polypeptides, GFD/FcK and SMB/FcH, with the following amino acid composition (N to C-terminal, IUPAC):

GFD/FcK
(SEQ ID No. 6)
SNELHQVPSNCDCLNGGTCVSNKYFSNIHWCNCPKKFGGQHCEIDKSKGSGLELE
VLFQGPIE

[symbol]

[symbol]

[symbol]

[symbol]

[symbol]

[symbol]

[symbol]

[symbol]

[symbol]

[symbol]

[symbol]

[symbol]

### Composed of:

In bold: Human uPA (amino acids 1-48)* (corresponding to SEQ ID No. 1),
in cursive: Artificial linker region ** and
in bold cursive: Human immunoglobulin (IgG) hinge and constant region (Fc) containing the “Knob” substitution (Y407T, underlined)***
*Shorter and longer pieces of uPA may also work. A sequence comprising amino acids 11-42 (underlined) may represent the minimal functional sequence.
**Different length and sequences of the linker regions may work equally well or better. The minimal length may be zero (i.e. no linker).
***The “Knob” substitution is utilized in this study to increase the quantity of heterodimers formed during expression but is not predicted to have any effect on the inhibitory quality of the heterodimer.

SMB/FcH
(SEQ ID No. 7)
DQESCKGRCTEGFNVDKKCQCDELCSYYQSCCTDYTAECKPGSGLELEVLFQGPIE

[symbol]

[symbol]

[symbol]

[symbol]

[symbol]

[symbol]

[symbol]

[symbol]

[symbol]

[symbol]

[symbol]

[symbol]

[symbol]

.

### Composed of:

In bold: Human VN (amino acids 141)* (corresponding to SEQ ID No. 2),
in cursive: Artificial linker region** and
in bold cursive Human immunoglobulin (IgG) hinge and constant region (Fc) containing the “Hole” substitution (T366Y, underlined)***
*Shorter and longer pieces of VN may also work. A sequence comprising amino acids 5-39 (underlined) may represent the minimal functional sequence.
**Different length and sequences of the linker regions may work equally well or better. The minimal length may be zero (i.e. no linker).
***The “Hole” substitution is utilized in this study to increase the quantity of heterodimers formed during expression but is not predicted to have any effect on the inhibitory quality of the heterodimer.

### Expression and Purification of Recombinant Proteins

Phoenix cells (semi-confluent 10 cm plates) were washed once with pre-warmed serum-free DMEM and added 8 ml of OptiMEM (Invitrogen). For uPAR-lock (i.e. GFD/SMB-FcK/H) cultures were transfected with a mixture of pN1-GFD/FcK and pN1-SMB/FcH (3+3 μg/10 plate) using the Fugene transfection reagent (Roche) according to the manufactures instructions. The control proteins GFD/GFD and SMB/SMB were expressed by co-transfecting pN1-GFD/hFcK+pN1-GFD/hFcH and pN1-SMB/hFcK+pN1-SMB/hFcH, respectively. The transfected cultures were left for 6-8 days after which supernatants were collected and filtered (0.45 μm). Proteins were purified on Protein A Sepharose, eluted using 0.1 M Glycine pH 2.8, 0.5 M NaCl and dialyzed extensively against PBS.

### In Vitro Binding Assays

96-well immuno plates (NUNC MaxiSorb, blackwell) were coated with pro-uPA or VN (100 μl, 10 nM) diluted in coating buffer (50 mM sodium carbonate, pH 9.6) at 4° C. ON. Plates were washed with wash buffer (phosphate buffered saline (PBS) containing 0.1% Tween-20 (PBS-T)) and non-specific binding sites blocked (0.15 ml/well) with blocking buffer (PBS containing 2% bovine serum albumin (BSA)) for 1-2 hour at RT. After washing with PBS-T, wells were incubated with uPAR/mFc (1 nM) in the presence or absence of the agonists and antagonists to be tested prepared in dilution buffer (PBS containing 1% BSA). The binding was allowed to occur for 1-2 hours at RT and the plates washed three times with wash buffer. Bound uPAR/mFc was detected by sequential incubations with a biotinylated goat anti-mouse Fc antibody (Sigma) and Eu³⁺ labeled streptavidin (Perkin Elmer). Bound Eu³⁺ was quantified by dissociation-enhanced time-resolved fluorescence measurement using an Envision Xcite plate reader (Perkin Elmer) using the DELFIA protocol.

### Cell Lines

The 293/uPAR, 293/uPAR^{T54A} and 293/mock cell lines were generated by stable transfection of HEK293 Flp-In T-REx cells (Invitrogen Corp.) with the pcDNAS/FRT-TO expression vector containing a wild-type human uPAR cDNA (293/uPAR), an uPAR cDNA containing the Thr54Ala substitution (293/uPAR^{T54A}) or the empty expression vector (293/mock) as described in detail previously (Madsen et al. 2007).

### Adhesion Assays

Cells were seeded in the wells of E-Plates (Roche) and cell adhesion was monitored at regular intervals using a real time cell analyzer (RTCA, xCELLigence SP, Roche). The RTCA system measures the electrical impedance across interdigitated microelectrodes integrated on the bottom of tissue culture 96-well E-Plates. The presence of the cells on top of the electrodes affect the local ionic environment at the electrode/solution interface, leading to an increase in the electrode impedance. The stronger the cell adhesion the larger the increase in electrode impedance.

### Migration Assay

Time-lapse live-cell imaging was performed at 37° C., 5% CO₂ with an inverted Olympus IX80 microscope equipped with an incubation chamber (OKOlab) to control CO₂ and temperature. Cells were plated in 12 well plates (Nunc) at the confluence of 1×10⁵ cell/well. Time-lapse imaging was performed in serum-containing growth medium. Cells were viewed through 10× (uPlan FLN 10× Ph1, N.A. 0.30; Olympus) objective lenses and pictures were taken every 5 minutes for 5 h. The acquisition system includes a digital camera (Hamamatsu Orca-ER) and System Control Software Olympus ScanR. Adjustment of brightness/contrast, smoothening and sharpness of images was done using ImageJ 1.42q and always applied to the entire image. Cell migration speed was quantified with ImageJ 1.42q using the plug-in “manual tracking”. In the experiment randomly chosen cells were tracked and their average migration speed throughout the experiment calculated.

### Cloning of SMB-GFD/mFc and GFD-SMB/mFc

The expression vectors for recombinant proteins tagged with a mouse IgG constant region (pFRT/TO-mFc) was generated by replacing (Xho1/Not1) the human Fc region of pFRT/TO-Fc with the mouse Fc region taken from the uPAR/mFc expression vector. To generate the GFD-SMB chimera a human uPA cDNA was amplified with oligos ATFkpnF/GLINKR and a human VN cDNA with oligos SLINKF/SMBRV2. The two PCR products were purified and co-amplified with oligos ATFkpnF/SMBRV2. To generate the SMB-GFD chimera a human VN cDNA was amplified with oligos hVnUkpn/SLINKR and a human uPA cDNA with oligos GLINKF/GFDRV. The two PCR products were purified and co-amplified with oligos VnUkpn/GFDRV. The GFD-SMB and SMB-GFD chimeras were cloned Kpn1/Xho1 into pFRT/TO-mFc to generate expression vectors encoding GFD-SMB/mFc and SMB-GFD/mFc. The expression vector encoding the SMB-GFD chimera tagged with a human Fc (SMB-GFD/Fc, uPAR-lockV2), was generated by cloning the SMB-GFD chimera Kpn1/Xho1 into pFRT/TO-Fc.

### Expression and Purification of Recombinant Proteins

The pFRT/TO-GFD-SMB/mFc, pFRT/TO-SMB-GFD/mFc and pFRT/TO-SMB-GFD/Fc expression vectors were transfected into CHO Flp-In cells (Invitrogen Corp.) and the recombinant proteins expressed under serum-free conditions as previously described (Madsen et al., JCB 2007). The recombinant chimeras were purified from the conditioned media by standard Protein A affinity chromatography and dialyzed extensively against PBS.

Oligonucleotide sequences
SLINKF:
(SEQ ID No. 30)
5′-tcaggcggaggtggctctggcggtggcggacaagagtcatgcaagg
gc-3′,
GLINKR:
(SEQ ID No. 31)
5′-agagccacctccgcctgaaccgcctccaccggtttttgacttatct
at-3′,
SLINKR:
(SEQ ID No. 32)
5′-agagccacctccgcctgaaccgcctccaccgggcttgcactcagcc
gt-3′,
GLINKF:
(SEQ ID No. 33)
5′-tcaggcggaggtggctctggcggtggcggaccatcgaactgtgact
gt-3′.

### Xenograft Experiments

Six-week-old male Balb C nu/nu mice were obtained from Charles River. Before inoculation, PC-3 cells growing in serum-containing medium were washed with phosphate buffered saline (PBS), harvested by trypsinization, and pelleted at 1200 rpm for 7 minutes. Cell (1.0×10⁶) were resuspended in 200 μl of PBS with 20% Matrigel. Animals were anesthetized by intraperitoneal (i.p) injection of Avertin and 1.0×10⁶ cells were inoculated subcutaneously (s.c.) using a 26-gauge needle into the right flank of anesthetized mice. 5 days after xenografting the animals were randomized into 2 control groups, where animals were treated twice a week i.p. with vehicle (n=5, PBS) non-immune mouse IgG1 (n=5, 10 mg/kg), and an experimental group, where animals were treated with uPAR-lock V2 (n=5, 10 mg/kg). The animals were monitored twice a week for 7 weeks for tumor development and growth. Tumor volume was determined according to the formula: tumor volume=shorter diameter²×longer diameter/2. Two mice, that did not develop palpable tumors, (one from the IgG control group and one from uPAR-lock V2 group) were excluded from the analysis. There was no significant difference between tumor growth in PBS and IgG treated animals (data not shown) and the data from these mice were pooled (n=9) for the comparison with the experimental uPAR-lock V2 group (n=4). Results were analyzed as the mean±SE, and comparisons of the experimental data were analyzed by unpaired, two-tailed, equal variance, t-test.

Composition of GFD-SMB/mFc, SMB-GFD/mFc and SMB-GFD/hFc (uPAR-LockV2)

The composition of the above constructs is a molecule, in particular a disulphide-linked homodimer composed of the two polypeptides GFD-SMB/mFc or SMB-GFD/mFc or SMB-GFD/hFc, with the following amino acid composition (N to C-terminal, IUPAC):

GFD-SMB/mFc
(SEQ ID No. 34)
SNELHQVPSNCDCLNGGTCVSNKYFSNIHVVCNCPKKFGGQHCEIDKSKTGGGGSGGG
GSGGGGQESCKGRCTEGFNVDKKCQCDELCSYYQSCCTDYTAECKPGSGLEAGA
GPRDCGCKPCICTVPEVSSVFIFPPKPKDVLTITLTPKVTCVVVDISKDDPEVQFSWFVDDV
EVHTAQTKPREEQFNSTERSVSELPIMHQDWLNGKEFKCRVNSAAFPAPIEKTISKTKGRP
KAPQVYTIPPPKEQMAKDKVSLTCMITDFFPEDITVEWQWNGQPAENYKNTQPIMDTDGS
YFVYSKLNYQKSNWEAGNTFTCSVLHEGLHNHHTEKSLSHSPGK.

GFD-SMB/mFc is composed of residues 1-49 of human uPA (GFD, plain text), a GGGGSGGGGSGGGG (SEQ ID No. 3) linker (underlined), residues 2-41 of human VN (SMB, in bold), a GSGLEAGAG (aa 104-112 of SEQ ID No. 34) linker (underlined cursive) and the heavy chain constant region from a mouse immunoglobulin (mFc, cursive).

SMB-GFD/mFc
(SEQ ID No. 35)
DQESCKGRCTEGFNVDKKCQCDELCSYYQSCCTDYTAECKPGGGGSGGGGSGGGGP
SNCDCLNGGTCVSNKYFSNIHWCNCPKKFGGQHCEIDKSKGSGLEAGAGPRDCGC
KPCICTVPEVSSVFIFPPKPKDVLTITLTPKVTCVVVDISKDDPEVQFSWFVDDVEVHTAQT
KPREEQFNSTFRSVSELPIMHQDWLNGKEFKCRVNSAAFPAPIEKTISKTKGRPKAPQVYTI
PPPKEQMAKDKVSLTCMITDFFPEDITVEWQWNGQPAENYKNTQPIMDTDGSYFVYSKLN
VQKSNWEAGNTFTCSVLHEGLHNHHTEKSLSHSPGK.

SMB-GFD/mFc is composed of residues 1-41 of human VN (SMB, plain text), a GGGGSGGGGSGGGG (SEQ ID No. 3) linker (underlined), residues 8-48 of human uPA (GFD, in bold), a GSGLEAGAG (aa 104-112 of SEQ ID No. 34) linker (underlined cursive) and the heavy chain constant region from a mouse immunoglobulin (mFc, cursive).

SMB-GFD/hFc (uPAR-LockV2)

(SEQ ID No. 8)

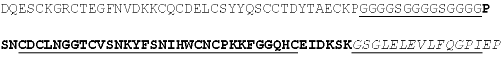

KSCDKTHTCPPCPAPELLGGPSVFLEPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPENNYKATPPVLDS
DGSFFLYSKLTVDKSRWQQGNVFSCSVMHEALHNHYTQKSLSLSPGK

GFD-SMB/hFc (uPAR-lockV2) is composed of residues 1-41 of human VN* (SMB, plain text) (corresponding to SEQ ID No. 2), a GGGGSGGGGSGGGG (SEQ ID No. 3) linker **(underlined), residues 8-48 of human uPA***(GFD, in bold) (corresponding to aa 8-48 of SEQ ID No. 1), a GSGLELEVLFQGPIE (SEQ ID No. 4) linker **(underlined cursive) and the heavy chain constant region from a human immunoglobulin (hFc, cursive).
*Shorter and longer pieces of VN may also work. The sequence consisting of amino acids 5-39 (grey shadow) may represent the minimal functional sequence.
**Different length and sequences of the linker regions may work equally well or better. The minimal length may be zero (i.e. no linker).
***Shorter and longer pieces of uPA may also work. A sequence comprising amino acids 11-42 (bold and underlined) may represent the minimal functional sequence.

### Immunohistochemical Analyses

For immunohistochemical analysis, primary tumors were excised, fixed in 4% paraformaldehyde (Formalin) and embedded in optimal cutting temperature (OCT) resin (Killik, BIO-OPTICA). Tissue blocks were sectioned at 8 μm and mounted onto positively charged glass slides for immuno-staining. For Ki-67 staining, sections were incubated with acetone at 4° C. for 1 minute. Slides were washed with PBS followed by blocking in pre-incubation buffer (PBS with 6% BSA and 10% FBS) for 1 h at RT. Slides were incubated with Ki-67 antibody (diluted 1:500) overnight at 4° C. followed by washing with PBS. For detection anti-rabbit Cy3 (1:200) and DAPI (1:2500) were used. Slides were mounted with Vectamount AQ. For detection of apoptotic cells, sections were incubated with 80% ethanol at room temperature for 1 minute. Slides were washed with PBS followed by blocking in pre-incubation buffer for 1 h at RT. Primary antibody (Cleaved caspase-3, 1:200) incubation was done overnight at 4° C. followed by washing with PBS. Detection was done as for Ki-67 stained slides. For quantification of cell proliferation and apoptosis, a total of 24 sections per animal were analyzed at 10× magnification, respectively. Data are shown as the average number of positive cells per field.

For tumor imaging experiments, uPAR-lockV2 and mouse IgG were labeled with Alexa488 dye according to the manufactures instructions. Mice carrying PC-3 tumors (8 weeks post xenografting) were injected with 50 μg labeled protein via intraperitoneal route and the tumors harvested 24 hours later. Tumor tissues were processed as described for immunohistochemical analysis.

### Results

Rationale of uPAR-Lock.

The growth factor-like domains of uPA (GFD) and the somatomedin B domain of VN (SMB) contains all the uPAR-binding determinants of the intact molecules, but lack their biological activity in extracellular proteolysis and cell signaling. Consequently, these domains are specific competitive antagonists of the uPAR:uPA and uPAR:VN interactions, respectively. Complete inhibition of both the uPA and VN mediated biological activities of uPAR requires the occupancy of both binding sites on the receptor and this may be achieved using the isolated GFD and SMB domains as illustrated in FIG. 1A. Inhibition requires two consecutive first-order inter-molecular binding reactions (1 and 2) a may follow two routes (A or B) depending on which ligand binds the receptor first. The efficacy of uPAR-inhibition in vivo by this approach is limited by the affinities of the different interactions and therefore upon concentration of GFD and SMB that can be achieved and maintained in vivo. As the GFD and SMB domains are rather small they are very likely to be rapidly cleared from the circulation. As the SMB domain has an affinity for uPAR (K_{D}=360 nM) that is about 1000-fold weaker than the affinity of GFD for the receptor (K_{D}=0.29 nM) (Gardsvoll & Ploug, 2007) the binding of this domain is particularly likely to limit the efficacy of this approach.

To significantly improve the efficacy of using GFD and SMB to block uPAR-function, the author hypothesized that forcing proximity between the two domains by attachment to a common scaffold may result in a compound (named uPAR-lock) with vastly improved inhibitory properties as compare to the isolated domains (FIG. 1B). In uPAR-lock, since the GFD and SMB domains are located on a common scaffold, the second binding reactions (A2 and B2), which are essential for inhibition, are now a zero-order intra-molecular rearrangement (i.e. concentration independent). As this second binding reaction is expected to be several orders of magnitude more efficient than intermolecular binding reactions the inhibitory activity of uPAR-lock will only be limited by the initial binding (reactions A1 or B1) and how well the scaffold is designed. Consequently, uPAR-lock is a stronger uPA-antagonist than GFD (similar on-rate, reduced off-rate) and a much stronger VN-antagonist (>1000-fold) than SMB (similar on-rate, dramatically decreased off-rate).

The GFD/SMB-scaffold may be generated in different ways as shown in FIG. 1C. The GFD and SMB domains may be attached to immunoglobulin heavy chain constant regions (Fc) and covalent heterodimers isolated. The desired GFD/SMB-scaffold may also be generated using peptide sequences that form dimers like leucine zippers. Finally the GFD and SMB domains may be expressed as a single polypeptide containing appropriate linkers. As an example, in the present study the immunoglobulin scaffold was used. Longer or shorter pieces of GFD and SMB may work as well. Moreover GFD and SMB may be derived from orthologue genes. Changing the length and sequence of the linker connecting the GFD and SMB to the scaffold may further improve the activity. Mutations in SMB and GFD that improve uPAR-binding may also be introduced. The leucine zipper and single peptide scaffold approaches are also suitable. A “uPAR-lock” does not necessarily need to be based on the GFD and SMB domains. One or both of these building blocks may be replaced with other uPAR-binding domains (like antibody fragments) or peptides that bind to uPAR at sites identical to, or overlapping with, the uPA-binding site and/or the VN-binding site.

Construction of an uPAR-Lock

Inspection of the crystal structure of the ternary complex between uPAR, the aminoterminal fragment of uPA (ATF) and the somatomedin B domain of VN (Huai et al, 2008) reveals that the peptide backbone of uPA and VN are closely located at some positions. In particular Lys48 in uPA and Pro41 in VN are only distant about 19 Å (FIG. 2A). The peptide backbone polarities of uPA and VN at these positions are approximately parallel, pointing outwards from the receptor and away from the membrane anchorage location. Connecting residues 1-48 of uPA (i.e. GFD) and 1-41 of VN (i.e. SMB) to a common scaffold, via their C-termini, is thus predicted to generate a chimera that favors the contemporary and constructive binding of both domains to uPAR.

To join GFD and SMB onto a common scaffold the author choose the constant region (Fc) of human IgG as these form stable dimers with the two N-termini located in proximity (FIG. 2B). For GFD the author constructed an expression vector encoding a mature polypeptide (named GFD/Fc) composed of amino acids 1-48 of human uPA (SNELHQVPSNCDCLNGGTCVSNKYFSNIHWCNCPKKFGGQHCEIDKSK (SEQ ID No. 1)), a short linker region containing a PreScission protease cleavage site (GSGLELEVLFQGPIE (SEQ ID No. 4)) and the hinge and constant regions (Fc) of a human IgG1 immunoglobulin heavy chain (PKSCDKTHTCPPCPAPELLGGPSVFLFPPKPKDTLMISRTPEVTCVVVDVSHEDPEVK FNAWYVDGVEVHNAKTKPREEQYNSTYRVVSVLTVLHQDWLNGKEYKCKVSNKALP APIEKTISKAKGQPREPQVYTLPPSRDELTKNQVSLTCLVKGFYPSDIAVEWESNGQPE NNYKATPPVLDSDGSFFLYSKLTVDKSRWQQGNVFSCSVMHEALHNHYTQKSLSLSP GK (SEQ ID No. 5 wherein Xaa in position 150 is T and Xaa in position 191 is Y)). The corresponding SMB/Fc polypeptide have amino acids 1-41 of human VN (DQESCKGRCTEGFNVDKKCQCDELCSYYQSCCTDYTAECKP (SEQ ID No. 2)) and identical linker and Fc regions (Fc regions are identical, except for the Knob and Hole mutations).

To favor hetero-dimerization between the Fc-tagged GFD and SMB the author modified the Fc-regions to carry either “knob” (T366Y, FcK) or “hole” (Y407T, FcH) substitutions as described previously (Ridgway et al, 1996). Co-expression in mammalian cells of the chimeric proteins GFD/FcK and SMB/FcH results in the predominant (>90%) production of GFD/FcK-SMB/FcH hetero-dimers (i.e. uPAR-lock) as well as minor quantities (total <10%) of the (GFD/FcK)₂ and (SMB/FcH)₂ homo-dimers and GFD/FcK and SMB/FcH monomers.

uPAR-Lock Binds uPAR Specifically

To confirm the receptor binding activity of uPAR-lock, immobilized soluble uPAR were incubated with a dilution series of conditioned medium of Phoenix cells co-transfected with equal amounts of GFD/FcK and SMB/FcH expression vectors. After washing, bound uPAR-lock was detected using sequential incubations with a biotinylated anti-human Fc antibody and Eu-labeled streptavidin. As shown in FIG. 3A, the conditioned medium of uPAR-lock transfected cells indeed contains specific and dose-dependent uPAR-binding activity.

Purification of uPAR-Lock

To better characterize uPAR-lock the protein was purified from the conditioned medium of transfected cells by standard Protein A affinity chromatography. When analyzed by SDS-PAGE (FIG. 3B) under non-reducing conditions uPAR-lock has an apparent molecular weight of ˜75 kDa which is in good agreement with the molecular weight predicted from the amino acid composition. Under reducing conditions two major peptides (40 and 45 kDa) are observed representing the SMB/FcH and GFD/FcK monomers. Because of the small difference in molecular weight between these it is, however, not possible to reliably estimate the efficiency of hetero-dimerization.

uPAR-Lock is a Competitive Antagonist of the uPA/uPAR-Interaction

To test the activity of uPAR-lock as an antagonist of the uPAR:uPA interaction the author utilized a binding assay in which purified uPAR tagged with a mouse Fc (uPAR/mFc) is allowed to bind to immobilized uPA (FIG. 4A). In this assay uPAR/mFc bind uPA in a saturable manner and with high affinity. To determine the antagonistic properties of uPAR-lock the author used the same assay with a fixed concentration of uPAR/mFc (5 nM) mixed with increasing concentrations of uPAR-lock or uPA (FIG. 4B). In this assay, uPAR-lock and uPA inhibit uPAR/mFc binding to immobilized uPA with a very similar dose response (uPA IC50=0.21 nM, uPAR-lock IC50=0.31 nM) documenting that uPAR-lock is indeed an efficient competitive antagonist of the uPAR:uPA interaction.

uPAR-Lock is a Potent Antagonist of the uPAR/VN-Interaction

To test the activity of uPAR-lock as an antagonist of the uPAR:VN-interaction the author utilized a binding assay in which uPAR/mFc is allowed to bind immobilized VN in the presence of uPA (FIG. 4C). In this assay uPAR/mFc binds to immobilized VN with high affinity in an uPA-dependent manner. To test the activity of uPAR-lock the author conducted the experiment with a fixed concentration of uPAR/mFc (1 nM) and an excess of uPA (5 nM) mixed with increasing concentrations of uPAR-lock or uPA for comparison (FIG. 4D). In this assay uPAR-lock is a very potent inhibitor of uPAR-binding to immobilized VN (IC50=0.22 nM, 95% confidence interval 0.19-0.25 nM). This is in sharp contrast to uPA, which is required for uPAR-binding to VN (FIG. 4C) and fail to inhibit the interaction noticeable even if present in excess (FIGS. 4C and 4D). These experiments show that uPAR-lock is a potent antagonist of the uPAR:VN-interaction in vitro.

uPAR-Lock is a Potent Functional Inhibitor of uPAR-Mediated Cell Adhesion to VN

To evaluate the activity of uPAR-lock in inhibiting uPAR-signaling in live cells the author quantified cell adhesion to VN coated wells by impedance measurements using a real-time cell analyzer (RTCA, xCELLigence SP, Roche) (FIG. 5). In these experiments the author utilized 293 cells expressing a conditional uPAR mutant (uPAR^{T54A}, (Madsen et al, 2007)), which display very low VN-binding activity in the absence of uPA and full activity in the presence of uPA. When 293/mock and 293/uPAR^{T54A} cells are seeded on VN they adhere weakly to the substrate as illustrated by the time-dependent increase in cell index that arrives at a plateau after 1-1.5 hours. Subsequent addition of uPA induces a rapid and strong increase in the adhesion of 293/uPAR^{T54A} cells (Madsen et al, 2007) as evidenced here by the dramatic increase in cell index (black line). Addition of uPA to control cells (293/mock) does not promote cell adhesion (Madsen et al, 2007) as these do not express uPAR and does consistently also not induce noticeable changes in the cell index (brown line). When 293/uPAR^{T54A} cells are seeded in the presence of uPAR-lock (red line) the initial phase of cell adhesion is similar to that of untreated cells, or mock-transfected cells, demonstrating that uPAR-lock does not influence basal integrin mediated cell adhesion. Importantly, the treatment with uPAR-lock almost completely abrogates the increase in 293/uPAR^{T54A} cell index induced by uPA addition. These data show that uPAR-lock is a potent and specific inhibitor of uPA-induced, uPAR mediated, cell adhesion to VN.

Forced Proximity Between GFD and SMB Contributes to the Activity of uPAR-Lock in Antagonizing the uPA: uPAR-Interaction

The rationale behind the construction of uPAR-lock predicts that the forced proximity generated between the GFD and SMB domains by attachment to a common scaffold is essential to its potent antagonistic activity. To address this prediction experimentally, the author constructed variants of uPAR-lock having identical scaffolds but carrying either two GFD domains (named GFD/GFD) or two SMB domains (named SMB/SMB) as shown in FIG. 6A. An SDS-PAGE gel of these proteins is shown in FIG. 6B. The binding of recombinant SMB/SMB and GFD/GFD proteins, and a 1:1 mixture of these (GFD/GFD+SMB/SMB), were compared with uPAR-lock for binding to immobilized uPAR (FIG. 6C). The 1:1 mixture of GFD/GFD and SMB/SMB is a particularly good control as this sample has the same quantitative composition of GFD, SMB and scaffold domains as uPAR-lock, but with the GFD and SMB located on separate scaffolds. With the exception of SMB/SMB, all the protein preparations displayed dose-dependent binding to uPAR with the uPAR-lock binding being the strongest. This is coherent with the prediction that it is the high-affinity interaction between GFD and uPAR that “drives” the interaction. The fact that the GFD/GFD+SMB/SMB mixture binds less well, as compared to uPAR-lock, supports the assumption that the SMB domain on uPAR-lock stabilize its interaction with uPAR. The same protein preparations were also tested for their activity in competing the binding of uPAR/mFc to immobilized uPA. Very similar results were obtained (FIG. 6D). In this assay, the GFD/GFD preparation was more active than uPAR-lock consistent with the fact that at equal concentrations of uPAR-lock (i.e. GFD/SMB) and GFD/GFD the latter protein contains twice the number of high-affinity uPAR binding domains (i.e. it is a divalent homodimer).

Forced Proximity Between GFD and SMB is Essential for the Functional Antagonistic Activity of uPAR-Lock on uPAR-Mediated Cell Adhesion

To determine the importance of close proximity between GFD and SMB for the inhibitory activity of uPAR-lock on uPAR-mediated cell adhesion to VN, the author measured the changes in impedance in the process of 293/uPAR (FIG. 7A) and 293/uPAR^{T54A} (FIG. 7B) cell adhesion to immobilized VN. In 293/uPAR cells, uPAR-lock (red line) reduces basal cell adhesion to VN as compared to untreated cells (black line) and abrogates the response to uPA. In the same cells, SMB/SMB (blue line) is essentially without activity while GFD/GFD (yellow line) and GFD/GFD+SMB/SMB (green line) display agonistic activity. In 293/uPAR^{T54A} cells, uPAR-lock is devoid of agonistic activity and completely block uPA-induced cell adhesion. In these cells, the agonistic effect of the GFD/GFD chimera and the GFD/GFD+SMB/SMB mixture is even more pronounced and the SMB/SMB chimera is inactive. These data very clearly demonstrate that the close proximity of the GFD and SMB domains in uPAR-lock are responsible and required for the inhibitory activity of the molecule on uPAR-signaling in cell adhesion to VN.

Forced Proximity Between GFD and SMB is Essential for the Functional Antagonistic Activity of uPAR-Lock on uPAR-Mediated Cell Migration

Downstream of cell adhesion, the expression of uPAR in 293 cells also stimulates random cell migration on serum-coated surfaces (Madsen et al, 2007). Consistently, the author found that uPAR-lock highly significantly inhibits basal migration of 293/uPAR cells (FIG. 8). This uPAR-lock activity requires the forced proximity between the SMB and GFD domains.

uPAR-Lock V2

Variants of uPAR-lock containing the SMB and GFD domains in a single polypeptide may possibly be generated and are likely to have several advantages. Firstly, the manufacture of these is less complicated as only a single polypeptide has to be expressed. Secondly, the number of SMB and GFD domains is always equal when present in a single polypeptide chain preventing the formation of undesired, non-inhibitory and/or agonistic variants like the SMB/Fc and GFD/Fc which will be present in low levels in uPAR-lock preparations requiring heterodimerization.

With this goal in mind, we constructed chimeras in which the SMB and GFD domains are engineered into a single polypeptide chain and tagged with a C-terminal mouse Fc tag. Two variants were made. The first (SMB-GFD/mFc) is composed of an N-terminal SMB domain, a linker, the GFD domain and a C-terminal mouse immunoglobulin constant region (mFc). The second (GFD-SMB/mFc), has a similar organization but the relative positions of the SMB and GFD domains are inverted. A cartoon illustrating the structures of these molecules is shown in FIG. 9A. These homodimeric proteins were expressed in CHO cells and purified by protein A affinity chromatography. To determine the functional inhibitory activity of GFD-SMB/mFc and SMB-GFD/mFc, we measured their potency in inhibiting uPAR-mediated cell adhesion to VN (FIG. 9, panels B and C). As it is evident from FIG. 9B, the SMB-GFD/mFc chimera induced a rapid dose and time dependent reduction in the adhesion of uPAR-expressing 293 cells to VN. The dose-response curves at one hour of treatment with SMB-GFD/mFc, GFD-SMB/mFc and uPAR-lock are shown in FIG. 9C and demonstrates that the SMB-GFD/mFc chimera (IC50=6.3 nM) is about 3-fold more potent than uPAR-lock (IC50=17 nM) or the GFD-SMB/mFc chimera (IC50=20 nM). As these reagents are made with the purpose of human use, we re-cloned the SMB-GFD chimera to have a human Fc-tag and termed this compound uPAR-lockV2. These data show that functional uPAR-lock variants having the SMB and GFD domains located in the same polypeptide chain may be generated and that this reorganization of the effector domains (i.e. the SMB and GFD) may even result in more potent inhibitors. Furthermore, the experiments documents that a large degree of flexibility is allowed for when generating uPAR-lock like chimeras.

As evident from the experiment shown in FIGS. 9B and 9C, the maximal inhibition of 293/uPAR cell adhesion to VN by uPAR-lock variants is incomplete with a maximal inhibition of about 60%. This residual cell adhesion is likely caused by integrin mediated cell adhesion to VN, which cannot be inhibited by uPAR-lock. To address this possibility directly we repeated the adhesion experiments substituting the VN coating with a recombinant protein VN(1-66)/Fc^{RAD} (Madsen et al JCB 2007) that is fully active in uPAR-binding, but deficient in integrin binding due to a single amino acid (G46A) substitution in the ⁴⁵RGD⁴⁷ sequence of VN. As can be seen in FIG. 10B, uPAR-lockV2 inhibits 293/uPAR cell adhesion almost completely while residual adhesion (about 20%) is still observed for uPAR-lock event at the highest tested concentration. Also the IC50 of uPAR-lockV2 (0.59 nM) is superior to that of uPAR-lock (2.3 nM).

Together these data show that uPAR-lockV2 is superior to uPAR-lock

### Proof of Concept

Inhibition of uPAR-function by uPAR-lock and uPAR-lockV2 is predicted to require the contemporary binding of both the SMB and GFD domains to the receptor and this was largely documented for uPAR-lock in FIG. 7. To demonstrate this point in a more elegant and conclusive way we generated variants of uPAR-lock and uPAR-lock V2 containing a single amino acid substitution in the SMB domain known to block the interaction with uPAR (Okumura Y, et al. J Biol Chem 2002). The domain structure of these variants, uPAR-lock^{D22A} and uPAR-lockV2^{D22A}, is illustrated in FIG. 10A and is identical to that of uPAR-lock and uPAR-lockV2, except for the single Asp22Ala substitution in the SMB domain. When assayed for biological activity in cell adhesion assays (FIG. 10C) both uPAR-lock^{D22A} and uPAR-lockV2^{D22A} are devoid of inhibitory activity and in fact displays dose-dependent agonistic activity. Together, these data demonstrate that the presence of functional GFD and SMB domains on a single scaffold is a fundamental requirement to uPAR-lock-like inhibitors of uPAR function.

uPAR-Lock V2 Inhibits Tumor Growth In Vivo

To determine the potential anti-tumor activity of uPAR-lock in vivo we conducted studies using a prostate cancer xenograft model. In this model, one million PC3 cells were inoculated in the right flank of male Balb C nu/nu mice through subcutaneous route. The xenografted animals were treated bi-weekly with uPAR-lockV2 by intraperitoneal injections and the volume of the tumors monitored by calibration. As shown in FIG. 11, the animals treated with uPAR-lockV2 displayed significantly reduced tumor volumes as compared to control animals. Treated animals displayed approximately 50% reduction in tumor volume and this is better than what has been observed by others using an inhibitory anti-uPAR antibody ATN-658 (Rabbani S A, et al. Neoplasia 2010).

Reduced Cell Proliferation and Enhanced Apoptosis in PC-3 Tumors of Animals Treated with uPAR-Lock

To investigate the biological reason for the reduced PC-3 tumor growth in animals treated with uPAR-lock we conducted immunohistochemistry analysis of sections of tumors taken from animals 8 weeks after xenografting (FIG. 12). To evaluate tumor cell proliferation, we stained for the proliferating cell antigen Ki-67 and to evaluate apoptosis we stained for activated (cleaved) caspase-3. As shown in FIG. 12A, tumors taken from mice treated with uPAR-lockV2 display a strong increase in the number of cells undergoing apoptosis as evidenced by cleaved caspase-3 reactivity and a marked decrease in the number of proliferating cells as marked by Ki-67 positivity. A quantification of these data is shown in FIG. 12B. Same results were obtained also with uPAR-lock. Together these data suggest that uPAR-lock suppresses tumor growth by promoting apoptosis and by reducing cell proliferation.

Tumor Imaging and Drug-Delivery Using uPAR-Lock

In addition to its direct activity in reducing cancer growth, uPAR-lock may potentially also be used for the imaging tumors and/or as a drug delivery vehicle. To address this possibility directly, we xenografted mice with PC-3 cells and allowed the primary tumors to grow for 8 weeks without any pharmacological intervention. We then injected tumor-bearing animals with 50 micrograms of Alexa488-labeled uPAR-lockV2 or Alexa488-labeled mouse IgG via intraperitoneal route. Twenty-four hours after the injection of labeled proteins the tumors were excised, fixed, embedded, sectioned and inspected by microscopy for the presence of green fluorescence signal in the tumor tissue. As shown in FIG. 13, tumors from mice injected with labeled uPAR-lock displayed zones of marked fluorescence, while no similar fluorescence was observed in animals receiving labeled IgG. Same results were obtained also with uPAR-lock. Although the cellular nature of these zones is unknown, the data clearly show that uPAR-lock is capable of targeting fluorescent dyes to the tumor tissue thus demonstrating that uPAR-lock may potentially be used for tumor imaging and/or as a drug delivery vehicle.

### REFERENCES

Adetugbo K (1978) Evolution of immunoglobulin subclasses. Primary structure of a murine myeloma gammal chain. J Biol Chem 253(17): 6068-6075
Blasi F, Carmeliet P (2002) uPAR: a versatile signalling orchestrator. Nat Rev Mol Cell Biol 3(12): 932-943.
Gardsvoll H, Ploug M (2007) Mapping of the Vitronectin-binding Site on the Urokinase Receptor: involvement of a coherent receptor interface consisting of residues from both domain i and the flanking interdomain linker region. J Biol Chem 282(18): 13561-13572
Huai Q, Zhou A, Lin L, Mazar A P, Parry G C, Callahan J, Shaw D E, Furie B, Furie B C, Huang M (2008) Crystal structures of two human vitronectin, urokinase and urokinase receptor complexes. Nat Struct Mol Biol 15(4): 422-423
Madsen C D, Ferraris G M, Andolfo A, Cunningham O, Sidenius N (2007) uPAR-induced cell adhesion and migration: vitronectin provides the key. J Cell Biol 177(5): 927-939
Moll J R, Ruvinov S B, Pastan I, Vinson C (2001) Designed heterodimerizing leucine zippers with a ranger of pIs and stabilities up to 10(-15) M. Protein Sci 10(3): 649-655
Okumura Y, Kamikubo Y, Curriden S A, Wang J, Kiwada T, Futaki S, Kitagawa K, Loskutoff D J. (2002) Kinetic analysis of the interaction between vitronectin and the urokinase receptor. J Biol Chem. 2002 Mar. 15; 277(11):9395-404. Epub 2001 Dec. 31.
Rabbani S A, Ateeq B, Arakelian A, Valentino M L, Shaw D E, Dauffenbach L M, Kerfoot C A, Mazar A P. (2010) An anti-urokinase plasminogen activator receptor antibody (ATN-658) blocks prostate cancer invasion, migration, growth, and experimental skeletal metastasis in vitro and in vivo. Neoplasia. 2010 October; 12(10):778-88.
Ridgway J B, Presta L G, Carter P (1996) ‘Knobs-into-holes’ engineering of antibody CH3 domains for heavy chain heterodimerization. Protein Eng 9(7): 617-621
Smith H W, Marra P, Marshall C J (2008) uPAR promotes formation of the p130Cas-Crk complex to activate Rac through DOCK180. J Cell Biol 182(4): 777-790
Smith H W, Marshall C J (2010) Regulation of cell signalling by uPAR. Nat Rev Mol Cell Biol 11(1): 23-36

1. A dimeric molecule comprising two polypeptides selected from the group consisting of:
a first polypeptide comprising the growth factor-like domain of uPA (GFD domain) from aa. 11 to aa. 42 of SEQ ID No. 1, or a polypeptide encoded by the correspondent region from an uPA orthologous gene, or functional mutants or derivatives or analogues thereof; and
a second polypeptide comprising the somatomedin B domain of VN (SMB domain) from aa. 5 to aa. 39 of SEQ ID No. 2, or a polypeptide encoded by the correspondent region from a VN orthologous gene, or functional mutants or derivatives or analogues thereof;
wherein the two polypeptides are linked one to the other by a molecular scaffold.

2. The dimeric molecule of claim 1, wherein
the first polypeptide further comprises the SMB domain from aa. 5 to aa. 39 of SEQ ID No. 2, or a polypeptide encoded by the same region from a VN orthologous gene, or functional mutants or derivatives or analogues thereof; and
the second polypeptide further comprises the GFD domain from aa. 11 to aa. 42 of SEQ ID No. 1, or a polypeptide encoded by the same region from an uPA orthologous gene, or functional mutants or derivatives or analogues thereof.

3. The dimeric molecule according to claim 1, wherein the GFD domain consists essentially of aa. 8 to aa. 48 of SEQ ID No. 1.

4. The dimeric molecule according to claim 1, wherein the GFD domain consists of aa. 1 to aa. 48 of SEQ ID No. 1.

5. The dimeric molecule according to claim 1, wherein the SMB domain consists of aa. 1 to 41 of SEQ ID No. 2.

6. The dimeric molecule according to claim 2, wherein the first and the second polypeptide:
a) comprise a sequence ordered as N-terminal-SMB domain-GFD domain-C-terminal; and
b) are linked to the molecular scaffold through their C-terminals.

7. The dimeric molecule according to claim 2, wherein the SMB domain and the GFD domain are linked by a first linker peptide.

8. The dimeric molecule according to claim 7 wherein the first linker peptide consists essentially of the sequence of SEQ ID No. 3.

9. The dimeric molecule of claim 1, wherein each of first and second polypeptide are linked to the molecular scaffold by means of a second linker peptide.

10. The dimeric molecule of claim 9 wherein the linker peptide consists essentially of the sequence of SEQ ID No. 4.

11. The dimeric molecule of claim 1, wherein the molecular scaffold is an immunoglobulin constant region (Fc), a leucine zipper, a chemical or a peptide linker.

12. The dimeric molecule of claim 11 wherein each of heavy chain constant regions of Fc has essentially the sequence of SEQ ID No. 5.

13. The dimeric molecule of claim 12 consisting essentially of a first monomer of sequence of SEQ ID No. 6, and of a second monomer of sequence of SEQ ID No. 7.

14. The dimeric molecule of claim 12 wherein the first and the second monomer have the sequence of SEQ ID No. 8.

15-16. (canceled)

17. The dimeric molecule according to claim 1 being conjugated to a therapeutic agent.

18. The dimeric molecule of claim 17 wherein the therapeutic agent is a radionuclide or a toxin.

19-20. (canceled)

21. Method of treatment of cancer comprising the administration to a subject in need thereof of a therapeutically effective amount of the dimeric molecule of claim 1.

22. A pharmaceutical composition comprising the dimeric molecule of claim 1 and appropriate diluents or excipients.

23. The pharmaceutical composition of claim 22 further comprising another therapeutic agent.

24. The pharmaceutical composition of claim 23 wherein the therapeutic agent is a radionuclide or a toxin.

25. A kit for the diagnosis of a uPAR-mediated pathology or tumor comprising the dimeric molecule of claim 1.


SEQUENCE LISTING (36 sequences)

SEQ ID NO: 1 - protein, 48 aa, Homo sapiens
SNELHQVPSNCDCLNGGTCVSNKYFSNIHWCNCPKKFGGQHCEIDKSK

SEQ ID NO: 2 - protein, 41 aa, Homo sapiens
DQESCKGRCTEGFNVDKKCQCDELCSYYQSCCTDYTAECKP

SEQ ID NO: 3 - protein, 14 aa, Artificial Sequence
  linker
GGGGSGGGGSGGGG

SEQ ID NO: 4 - protein, 15 aa, Artificial Sequence
  linker
GSGLELEVLFQGPIE

SEQ ID NO: 5 - protein, 231 aa, Artificial Sequence
  hFc constant region
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLXCLVKGFYPSDIAVEWESNGQPENNYKATPPVLDSDGSFFLXSKLTVDKSRWQQGNVFSCSVMHEALHNHYTQKSLSLSPGK

SEQ ID NO: 6 - protein, 294 aa, Artificial Sequence
  polypeptide GFD/FcK
SNELHQVPSNCDCLNGGTCVSNKYFSNIHWCNCPKKFGGQHCEIDKSKGSGLELEVLFQGPIEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKATPPVLDSDGSFFLYSKLTVDKSRWQQGNVFSCSVMHEALHNHYTQKSLSLSPGK

SEQ ID NO: 7 - protein, 287 aa, Artificial Sequence
  polypeptides SMB/FcH
DQESCKGRCTEGFNVDKKCQCDELCSYYQSCCTDYTAECKPGSGLELEVLFQGPIEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLTSKLTVDKSRWQQGNVFSCSVMHEALHNHYTQKSLSLSPGK

SEQ ID NO: 8 - protein, 342 aa, Artificial Sequence
  polypeptide SMB-GFD/hFc
DQESCKGRCTEGFNVDKKCQCDELCSYYQSCCTDYTAECKPGGGGSGGGGSGGGGPSNCDCLNGGTCVSNKYFSNIHWCNCPKKFGGQHCEIDKSKGSGLELEVLFQGPIEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 9 - protein, 431 aa, Homo sapiens
MRALLARLLLCVLVVSDSKGSNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADGKKPSSPPEELKFQCGQKTLRPRFKIIGGEFTTIENQPWFAAIYRRHRGGSVTYVCGGSLISPCWVISATHCFIDYPKKEDYIVYLGRSRLNSNTQGEMKFEVENLILHKDYSADTLAHHNDIALLKIRSKEGRCAQPSRTIQTICLPSMYNDPQFGTSCEITGFGKENSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKEENGLAL

SEQ ID NO: 10 - protein, 478 aa, Homo sapiens
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 11 - protein, 335 aa, Homo sapiens
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYRSGAAPQPGPAHLSLTITLLMTARLWGGTLLWT

SEQ ID NO: 12 - DNA, 31 bp, Artificial Sequence
  synthetic oligonucleotide
gtaaatgagcggccgcgtcgagtctagaggg

SEQ ID NO: 13 - DNA, 30 bp, Artificial Sequence
  synthetic oligonucleotide
ccctctagactcgacgcggccgctcattta

SEQ ID NO: 14 - DNA, 30 bp, Artificial Sequence
  synthetic oligonucleotide
tcgagctggaagttctgttccaggggccca

SEQ ID NO: 15 - DNA, 30 bp, Artificial Sequence
  synthetic oligonucleotide
agctacccggggaccttgtcttgaaggtcg

SEQ ID NO: 16 - DNA, 24 bp, Artificial Sequence
  synthetic oligonucleotide
cggggtaccatggcacccctgaga

SEQ ID NO: 17 - DNA, 28 bp, Artificial Sequence
  synthetic oligonucleotide
ttgcggccgctcatttacccggagacag

SEQ ID NO: 18 - DNA, 33 bp, Artificial Sequence
  synthetic oligonucleotide
aaccaggtcagcctgtactgcctggtcaaaggc

SEQ ID NO: 19 - DNA, 33 bp, Artificial Sequence
  synthetic oligonucleotide
gcctttgaccaggcagtacaggctgacctggtt

SEQ ID NO: 20 - DNA, 33 bp, Artificial Sequence
  synthetic oligonucleotide
ggctccttcttcctcaccagcaagctcaccgtg

SEQ ID NO: 21 - DNA, 33 bp, Artificial Sequence
  synthetic oligonucleotide
cacggtgagcttgctggtgaggaagaaggagcc

SEQ ID NO: 22 - DNA, 36 bp, Artificial Sequence
  synthetic oligonucleotide
gcggtacccgccaccatgagagccctgctggcgcgc

SEQ ID NO: 23 - DNA, 38 bp, Artificial Sequence
  synthetic oligonucleotide
gcctcgagtcctgatccttttgacttatctatttcaca

SEQ ID NO: 24 - DNA, 35 bp, Artificial Sequence
  synthetic oligonucleotide
gcctcgagtcctgatccgggcttgcactcagccgt

SEQ ID NO: 25 - DNA, 42 bp, Artificial Sequence
  synthetic oligonucleotide
gcgtcgacggtacccgccaccatgggtcacccgccgctgctg

SEQ ID NO: 26 - DNA, 30 bp, Artificial Sequence
  synthetic oligonucleotide
agcctcgagcccactgcggtactggacatc

SEQ ID NO: 27 - DNA, 58 bp, Artificial Sequence
  synthetic oligonucleotide
gcctcgaggggcccctggaacagaacttccagatccaggtctgggtggttacagccac

SEQ ID NO: 28 - DNA, 40 bp, Artificial Sequence
  synthetic oligonucleotide
gcctcgaggcaggagcaggacccagggattgtggttgtaa

SEQ ID NO: 29 - DNA, 28 bp, Artificial Sequence
  synthetic oligonucleotide
gcgcggccgctcatttaccaggagagtg

SEQ ID NO: 30 - DNA, 48 bp, Artificial Sequence
  synthetic oligonucleotide
tcaggcggaggtggctctggcggtggcggacaagagtcatgcaagggc

SEQ ID NO: 31 - DNA, 48 bp, Artificial Sequence
  synthetic oligonucleotide
agagccacctccgcctgaaccgcctccaccggtttttgacttatctat

SEQ ID NO: 32 - DNA, 48 bp, Artificial Sequence
  synthetic oligonucleotide
agagccacctccgcctgaaccgcctccaccgggcttgcactcagccgt

SEQ ID NO: 33 - DNA, 48 bp, Artificial Sequence
  synthetic oligonucleotide
tcaggcggaggtggctctggcggtggcggaccatcgaactgtgactgt

SEQ ID NO: 34 - protein, 338 aa, Artificial Sequence
  polypeptide GFD-SMB/mFc
SNELHQVPSNCDCLNGGTCVSNKYFSNIHWCNCPKKFGGQHCEIDKSKTGGGGSGGGGSGGGGQESCKGRCTEGFNVDKKCQCDELCSYYQSCCTDYTAECKPGSGLEAGAGPRDCGCKPCICTVPEVSSVFIFPPKPKDVLTITLTPKVTCVVVDISKDDPEVQFSWFVDDVEVHTAQTKPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 35 - protein, 331 aa, Artificial Sequence
  polypeptide SMB-GFD/mFc
DQESCKGRCTEGFNVDKKCQCDELCSYYQSCCTDYTAECKPGGGGSGGGGSGGGGPSNCDCLNGGTCVSNKYFSNIHWCNCPKKFGGQHCEIDKSKGSGLEAGAGPRDCGCKPCICTVPEVSSVFIFPPKPKDVLTITLTPKVTCVVVDISKDDPEVQFSWFVDDVEVHTAQTKPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 36 - protein, 14 aa, Artificial Sequence
  linker
LEVLFQGPLEAGAG